(19) United States
(12) Patent Application Publication
(10) Pub. No.: US 2023/0416381 A1
(43) Pub. Date: Dec. 28, 2023

(54) METHODS FOR TREATING OR PREVENTING ACUTE RESPIRATORY DISTRESS SYNDROME

(71) Applicant: CSL INNOVATION PTY LTD, Melbourne VIC (AU)

(72) Inventors: Catherine Mary OWCZAREK, Melbourne VIC (AU); Ian Keith CAMPBELL, Melbourne VIC (AU); Karolina KRSTEVSKI, Melbourne VIC (AU); Steven BOZINOVSKI, Melbourne (AU); Angel Francisco LOPEZ, Medindie (AU); Damon John TUMES, Flinders Park (AU)

(21) Appl. No.: 18/037,342

(22) PCT Filed: Dec. 20, 2021
(86) PCT No.: PCT/AU2021/051521

§ 371 Date: May 17, 2023

(30) Foreign Application Priority Data
Dec. 21, 2020 (AU) 2020904755
Oct. 20, 2021 (AU) 2021903362

(51) Int. Cl. C07K 16/28; A61K 45/06; A61P 37/06

(52) U.S. Cl. CPC C07K 16/2866; A61K 45/06; A61P 37/06; A61K 2039/505

(57) ABSTRACT

The present disclosure relates to methods of treating or preventing acute respiratory distress syndrome (ARDS) using compounds that bind to CD131 and neutralize signaling by IL-3, IL-5 and GM-CSF. The present disclosure also relates to compounds for use in the treatment or prevention of ARDS, as well as the use of such compounds in the manufacture of medicaments for the treatment or prevention of ARDS.

### RELATED APPLICATION DATA

The present application claims priority from Australian Patent Application No. 2020904755 filed on 21 Dec. 2020 entitled “Methods for treating or preventing acute respiratory distress syndrome” and Australian Patent Application No. 2021903362 filed on 20 Oct. 2021 entitled “Methods for treating or preventing acute respiratory distress syndrome”. The entire contents of both applications are hereby incorporated by reference.

### SEQUENCE LISTING

The present application is filed together with a Sequence Listing in electronic form. The entire contents of the Sequence Listing are hereby incorporated by reference.

### FIELD

The present disclosure relates to methods of treating or preventing acute respiratory distress syndrome.

### BACKGROUND

Acute respiratory distress syndrome (ARDS) is a severe, and often life-threatening complication of several systemic disorders and direct injury to the lungs. It is associated with a high mortality rate, primarily as a consequence of multiple organ failure. ARDS occurs when fluid builds up in the alveoli of the lungs, resulting in less oxygen reaching the bloodstream, which deprives organs of the oxygen they need to function. Symptoms of ARDS include severe shortness of breath, labored and unusually rapid breathing, low blood pressure, and confusion and extreme tiredness, which usually develop within a few hours to a few days after an original disease or trauma.

Despite decades of research and advances in medical technology, ARDS-associated mortality remains high, and no pharmacological therapies effectively improve its clinical course. For example, drug candidates that have failed in large trials include, at least, glucocorticoids, alprostadil, surfactant, ketoconazole, N-acetylcysteine, procysteine, lisofylline, and site-inactivated recombinant factor VIIa. The current standard of care is limited to supportive therapies, for example oxygenation, mechanical ventilation, fluid management, and prone positioning.

Therefore, there remains a need for new interventions for treating and preventing ARDS.

### SUMMARY

In producing the present invention, the inventors identified CD131 (the β common receptor) as a potential target for pharmacological intervention in ARDS. The inventors found that antibodies that bind to CD131, and inhibit signaling of interleukin (IL) 3, IL-5 and granulocyte-macrophage colony stimulating factor (GM-CSF), successfully reduced several measures of lung inflammation in animal models of ARDS. These findings provide the basis for methods of treating or preventing ARDS in a subject by administering compounds that bind to CD131 and/or inhibit signaling of IL-3, IL-5 and GM-CSF.

Accordingly, in an example, the present disclosure provides a method for treating or preventing ARDS in a subject, the method comprising administering one or more compound(s) that neutralize signaling by IL-3, IL-5 and GM-CSF to the subject. Similarly, the present disclosure provides one or more compound(s) that neutralize signaling by IL-3, IL-5 and GM-CSF for use in treating or preventing ARDS in a subject. The present disclosure also provides use of one or more compound(s) that neutralize signaling by IL-3, IL-5 and GM-CSF in the manufacture of a medicament for treating or preventing ARDS.
In one example, a compound binds to one or more of the following:
IL-5;
IL-3;
GM-CSF;
IL-5 receptor α (IL-5Rα);
IL-3 receptor α (IL-3Rα); and
GM-CSF receptor α (GM-CSFRα).

For example, the method may comprise administering a compound, e.g., an antibody that binds to IL-3 and neutralizes signaling by IL-3 and a compound, e.g., an antibody that binds to GM-CSF and neutralizes signaling by GM-CSF and a compound, e.g., an antibody that binds to IL-5 and neutralizes signaling by IL-5. Equally the method my comprise administering a compound, e.g., an antibody that binds to IL-3Rα and neutralizes signaling by IL-3 and a compound, e.g., an antibody that binds to GM-CSFRα and neutralizes signaling by GM-CSF and a compound, e.g., an antibody that binds to IL-5Rα and neutralizes signaling by IL-5. The method may alternatively comprise administering a combination of compounds one or more of which bind to one or more of the foregoing cytokines and one or more of which bind to one or more of the foregoing receptors.

In another example, the method comprises administering a multi-specific compound, e.g., antibody. For example, the multi-specific compound is trispecific and binds to IL-3 or IL-3Rα and inhibits signaling by IL-3 and binds to GM-CSF or GM-CSFRα and inhibits signaling by GM-CSF and binds to IL-5 or IL-5Rα and inhibits signaling by IL-5.

In another example, the method comprises administering a bispecific molecule, e.g., an antibody and a monospecific compound, e.g., antibody. For example, the bispecific molecule binds to IL-3 or IL-3Rα and inhibits signaling by IL-3 and binds to GM-CSF or GM-CSFRα and inhibits signaling by GM-CSF. In this case, the monospecific compound binds to IL-5 or IL-5Rα and inhibits signaling by IL-5. Other combinations of compounds will be apparent to the skilled person.

In another example, the present disclosure provides a method for preventing or treating ARDS in a subject, the method comprising administering a compound that binds to CD131 and neutralizes signaling by IL-3, IL-5 and GM-CSF to the subject.

The present disclosure also provides a compound that hinds to CD131 and neutralizes signaling by IL-3, IL-5 and GM-CSF to the subject for use in treating or preventing ARDS in a subject. The present disclosure also provides use of a compound that binds to CD131 and neutralizes signaling by IL-3, IL-5 and GM-CSF in the manufacture of a medicament for treating or preventing ARDS in a subject.

Compounds that bind to CD131 and inhibit IL-3, IL-5 and GM-CSF signaling can be used in accordance with the methods of the present disclosure to treat or prevent ARDS which is associated with any underlying condition. In some examples, the ARDS is associated with one or more of the following:
a) an infection;
b) inhalation or aspiration of a foreign substance;
c) a physical trauma; and
d) an inflammatory disease.

In one example, the ARDS is associated with a viral infection. In one example, the ARDS is associated with a bacterial infection. In one example, the ARDS is associated with a fungal infection. In one example, the ARDS is associated with sepsis.

In one example, the ARDS is associated with a coronavirus infection.

In one example, the ARDS is associated with a severe acute respiratory syndrome coronavirus (SARS-COV) infection. In one example, the ARDS is associated with a SARS-CoV-2 infection. Thus, in some examples, the subject has coronavirus disease 2019 (COVID-19). In particular, severe COVID-19 often results in ARDS. The methods of the present disclosure can be used to treat or prevent ARDS in severe COVID-19 subjects. Accordingly, in some examples, the subject has severe COVID-19.

In some examples, the ARDS is associated with inhalation or aspiration of a foreign substance. For instance, breathing high concentrations of smoke or chemical fumes can result in ARDS, as can aspirating vomit or near-drowning episodes.

In some examples, the ARDS is associated with severe pneumonia. Severe cases of pneumonia usually affect all five lobes of the lungs and can result in ARDS. Thus, in sonic examples, the subject has interstitial pneumonia.

In some examples, the ARDS is associated with a physical trauma. For example, head, chest and other major injuries can lead to ARDS. Accidents, such as falls or car crashes, can directly damage the lungs or the portion of the brain that controls breathing, thereby leading to ARDS. In some examples, the ARDS is associated with a lung injury. In some examples, the ARDS is associated with a brain injury. In some examples, the ARDS is associated with a burn injury.

In some examples, the ARDS is associated with surgery, such as cardiac surgery. For example, the subject being treated has undergone or is to undergo cardiac surgery, e.g., cardiopulmonary bypass surgery.

In some examples, the ARDS is associated with an inflammatory disease. For instance, pancreatitis can lead to ARDS as can other severe inflammatory diseases. In some examples, the ARDS is associated with a blood transfusion.

In some examples, the subject has ARDS.

In some examples, the subject satisfies the Berlin definition of ARDS. Thus, in some examples, the subject has:
a) an onset of ARDS within 1 week or less of clinical insult or initial respiratory symptoms;
b) an acute hypoxemic respiratory failure, as determined by a ratio of arterial partial pressure of oxygen to fraction of inspired oxygen (PaO₂/FiO₂ ratio) of 300 mmHg or less on at least 5 cm of continuous positive airway pressure (CPAP) or positive end expiratory pressure (PEEP),
c) bilateral opacities on chest radiographs not fully explained by effusions, consolidation, or atelectasis; and
d) respiratory failure not fully explained by cardiac failure or fluid overload.

In some examples of the methods of the disclosure, the ARDS is mild ARDS. In some examples, the ARDS is moderate ARDS. In some examples, the ARDS is severe ARDS. The severity of ARDS can be categorized according to the Berlin definition as follows:
(i) Mild ARDS: PaO₂/FiO₂ of 200-300 mmHg on at least 5 cm CPAP or PEEP;
(ii) Moderate ARDS: PaO₂/FiO₂ of 100-200 mmHg on at least 5 cm PEEP; and
(iii) Severe ARDS: PaO₂/FiO₂ of less than or equal to 100 mmHg on at least 5 cm PEEP.

Advantageously, the methods of the present disclosure can, in addition to treatment of existing ARDS, be used to prevent the onset of ARDS. Thus, in some examples, the subject does not have ARDS.

In some examples, the subject is at risk of developing ARDS. Methods of identifying subjects at risk of developing ARDS will be known by those skilled in the art and include those described herein.

In some examples, the subject has one or more or all of the following
a) a respiratory frequency of greater than 30 breaths per minute;
b) an oxygen saturation (SpO₂) of 93% or less on room air;
c) a ratio of arterial partial pressure of oxygen to fraction of inspired oxygen (PaO₂/FiO₂) of less than 300 mmHg;
d) a SpO₂/FiO₂ ratio of less than 218; and
e) radiographic lung infiltrates in an amount of greater than 50%.

The above criteria can, in some examples, be used to assess if a subject is at risk of developing ARDS.

In some examples, the subject is not receiving high flow oxygen therapy (HFOT) or non-invasive ventilation (NIV) at the time of administering the compound that binds to CD131.

In some examples, the compound that binds to CD131 is administered in an amount sufficient to reduce the severity of or prevent onset of one or more symptoms of ARDS.

In one example, the compound that binds to CD131 is administered in an amount sufficient to prevent endotracheal intubation or death prior to endotracheal intubation. Endotracheal intubation is a process of inserting a tube, i.e., an endotracheal tube, through the mouth of the subject and into the airways so that the subject can be placed on a mechanical. ventilator. Thus, the methods of the present disclosure additionally provide a method of preventing or reducing mechanical ventilation of a subject with ARDS, the method comprising administering a compound that binds to CD131 and neutralizes signaling by IL-3, IL-5 and GM-CSF to the subject.

In some examples, the compound that binds to CD131 is administered in an amount sufficient to achieve one or more or all of the following:
a) increase the subject's days alive and ventilator free;
b) decrease the subject's hospital length of stay (LOS);
c) improve the subject's clinical status as assessed on an 8-point National Institute of Allergy and Infectious Disease (NIAID) ordinal scale;
d) reduce or prevent use of continuous positive airway pressure (CPAP) or bilevel positive airway pressure (BiPAP);
e) reduce or prevent use of high-flow nasal cannula (HFNC);
f) reduce or prevent use of extracorporeal membrane oxygenation (ECMO); and
g) reduce or prevent an increase in the subject's Sequential Organ Failure Assessment (SOFA) score.

In some examples, the compound that binds to CD131 is administered in an amount sufficient to reduce, or prevent an increase in, inflammation in the subject's lungs. In one example, the compound that binds to CD131 is administered in an amount sufficient to enhance lung function.

In some examples, administration of the compound that hinds to CD131 reduces, or prevents an increase in, one or more or all of the following:
a) amount of neutrophils in the subject's blood,
b) amount of monocytes in the subject's blood,
c) neutrophil accumulation in the subject's lung,
d) macrophage accumulation in the subject's lung,
e) inflammation of the subject's lung,
f) oedema of the subject's lung,
g) NETosis in the subject's lung,
h) myeloperoxidase activity in the bronchoalveolar fluid (BALF) of the subject's lung,
i) amount of protein in the BALF of the subject's lung,
j) amount of dsDNA in the BALF of the subject's lung, and
k) a lung injury score in the subject.

In some examples, administration of the compound that binds to CD131 reduces, or prevents an increase in macrophage accumulation in the subject's lung. For example, the macrophages are alveolar macrophages, monocyte derived exudative macrophages and/or blood monocytes.

In some examples, administration of the compound that binds to CD131 reduces, or prevents an increase in eosinophil accumulation in the subject's lung.

In some examples the compound that binds to CD131 is administered in an amount sufficient to reduce, or prevent an increase in, total cell counts and/or total protein in BALF of the subject. In some examples, the compound that binds to CD131 is administered in an amount sufficient to reduce, or prevent an increase in, the level of neutrophils present in BALF of the subject.

In some examples, the compound that binds to CD131 is administered in an amount sufficient to reduce, or prevent an increase in, the level of neutrophil elastase and/or myeloperoxidase activity in BALF of the subject.

In some examples, the compound that binds to CD131 is administered in an amount sufficient to reduce, or prevent an increase in, the levels of any one or more or all of the following: G-CSF, plasminogen activator inhibitor-1 (PAI-1), D-dimer, neutrophil elastase, soluble receptor for AGE (sRAGE), interferon gamma (IFN-γ), interleukin 1β (IL-1β), IL-2, IL-4, IL-6, IL-8, IL-10, IL-12p70, IL-13, and tumor necrosis factor alpha (TNF-α). These biomarkers can be used to assess the efficacy of treatment or to assist in identifying a subject at risk of developing ARDS.

In some examples, the levels of the above proteins are reduced, or prevented from increasing, in the subject's lungs. In some examples, the levels of the above proteins are reduced, or prevented from increasing, in the subject's blood.

In some examples, the compound that hinds to CD131 is administered in an amount sufficient to reduce, or prevent an increase in, the levels of any one or more or all of the following: C—C motif chemokine ligand 2 (CCL2), CCL24 and IL-1α.

In some examples, the levels of the above proteins are reduced, or prevented from increasing, in the subject's lungs. In some examples, the levels of the above proteins are reduced, or prevented from increasing, in the subject's blood.

In some examples, the compound that binds to CD131 is administered in an amount sufficient to reduce, or prevent an increase in, the level of chemokine gene expression in the subject. For example, the compound that binds to CD131 is administered in an amount sufficient to reduce, or prevent an increase in, the level of expression of the monocyte/macrophage chemokine Ccl2 gene and/or the eosinophil chemokine Cc124 gene in the subject.

In some examples, the compound that binds to CD131 is administered in an amount sufficient to reduce, or prevent an increase in, the level of expression of inflammatory cytokine genes in the subject. For example, the compound binds to CD131 is administered in an amount sufficient to reduce, or prevent an increase in, the level of expression of the inflammatory cytokine Il1α a gene in the subject.

In some examples, the levels of expression of the above genes are reduced, or prevented from increasing, in the subject's lungs. In some examples, the levels of expression of the above genes are reduced, or prevented from increasing, in the subject's blood cells.

Furthermore, a person skilled in the art will appreciate that the terms “reduce” and “prevent an increase in” are used herein to refer to a lower amount of any of the items listed above, relative to either the amount in the subject prior to administration of the compound that binds to CD131, or relative to the amount in a corresponding control subject. For instance, the control subject may be a subject who receives a placebo and/or a standard of care therapy, rather than the compound that binds to CD131.

In some examples, the compound that binds to CD131 and neutralizes signaling by IL-3, IL-5 and GM-CSF prevents a decrease in percentage blood oxygenation in the subject.

In some examples, the compound that binds to CD131 and neutralizes signaling by IL-3, IL-5 and GM-CSF reduces a decrease in percentage blood oxygenation in the subject compared to a decrease observed in a subject to whom the compound has not been administered.

In one example, the percentage blood oxygenation in the subject administered a compound that binds to CD131 and neutralizes signaling by IL-3, IL-5 and GM-CSF is at least about 5%, or about 6%, or about 7%, or about 8%, or about 9%, or about 10%, or about 11%, or about 12%, or about 13%, or about 14%, or about 15% higher than the percentage blood oxygenation in a subject not administered the compound that binds to CD131 and neutralizes signaling by IL-3, IL-5 and GM-CSF.

In one example, the percentage blood oxygenation in the subject administered a compound that hinds to CD131 and neutralizes signaling by IL-3, IL-5 and GM-CSF is about 85%, or about 86%, or about 87%, or about 88%, or about 89%, or about 90%, or about 91%, or about 92%, or about 93%, or about 94%, or about 95%, or about 96%, or about 97%, or about 98%, or about 99%, or about 100%.

In one example, the percentage blood oxygenation in a subject administered a compound that binds to CD131 and neutralizes signaling by IL-3, IL-5 and GM-CSF is about 90%.

In one example, the percentage blood oxygenation in a subject administered a compound that binds to CD131 and neutralizes signaling by IL-3, IL-5 and GM-CSF is about 92%.

Methods for assessing each of the foregoing are known in the art and/or are described herein.

In some examples, the reduction in the amount of the item listed above, or prevention of an increase thereof, or the prevention of a decrease in percentage blood oxygenation is assessed within 30 days of first administration of the compound that binds to CD131. In some examples, the reduction in the amount of the item listed above, or prevention of an increase thereof, is assessed 1, 2, 3, 4, 5, 6, 7, 8, 9, 10, 14, 17, 21, 24 or 28 days after first administration of the compound that binds to CD131.

In one example, the compound that binds to CD131 and neutralizes signaling by IL-3, IL-5 and GM-CSF prevents an increase in levels of blood haemoglobin (HGB) in the subject.

In some examples, the compound that binds to CD131 and neutralizes signaling by IL-3, IL-5 and GM-CSF reduces an increase in levels of blood haemoglobin (HGB) in the subject compared to an increase observed in a subject to whom the compound has not been administered.

In one example, the compound that binds to CD131 inhibits GM-CSF-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.4 nM. In one example, the IC₅₀ is at least about 0.3 nM.

In one example, the compound that binds to CD131 inhibits IL-5-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 5 nM. For example, the IC₅₀ is at least about 4 nM. In one example, the IC₅₀ is at least about 4.5 nM or at least about 4.6 nM or at least about 4.7 nM. In one example, the IC₅₀ is at least about 4.6 nM.

In one example, the compound that binds to CD131 inhibits IL-3-induced proliferation of TF-1 cells with an IC₅₀ of at least 600 nM or 500 nM. For example, the IC₅₀ is at least about 400 nM. For example, the IC₅₀ is at least about 300 nM or 200 nM or 100 nM. For example, the IC₅₀ is at least about 50 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.2 nM or at least about 0.1 nM. In one example, the IC₅₀ is at least about 0.15 nM.

Methods for determining the IC₅₀ are described herein and include culturing TF-1 cells (e.g., about 1×10⁴ TF-1 cells) in the presence of the compound that binds to CD131. (e.g., for at least about 3 minutes or 1 hour, such as about 30 minutes) prior to adding the relevant growth factor (GM-CSF, IL-3 and/or IL-5) and culturing the cells further (e.g., for at least about 48 hours or at least about 72 hours or at least about 96 hours, e.g., for about 72 hours) and then determining cell proliferation. Cell proliferation can he determined by growing the cells in the presence of ³[H]-thymidine for about 6 hours and determining ³[H]-thymidine incorporation, e.g., by liquid-scintillation counting. By determining proliferation in a variety of concentrations of the compound that binds to CD131 an IC₅₀ can be determined.

In some examples, the compound that binds to CD131 inhibits
a) GM-CSF-induced proliferation of TF-1 cells with an IC50 of at least 100 nM; and/or
b) IL-5-induced proliferation of TF-1 cells with an IC50 of at least 100 nM; and/or
c) IL-3-induced proliferation of TF-1 cells with an IC50 of at least 100 nM.

In some examples, the compound that binds to CD131 inhibits
a) GM-CSF-induced proliferation of TF-1 cells with an IC50 of at least 50 nM; and/or
b) IL-5-induced proliferation of TF-1 cells with an IC50 of at least 50 nM; and/or
c) IL-3-induced proliferation of TF-1 cells with an IC50 of at least 50 nM.

In some examples, the compound that binds to CD131 inhibits
a) GM-CSF-induced proliferation of TF-1 cells with an IC50 of at least 10 nM; and/or
b) IL-5-induced proliferation of TF-1 cells with an IC50 of at least 10 nM; and/or
c) IL-3-induced proliferation of IF-1 cells with an IC50 of at least 10 nM.

In one example, the compound that binds to CD131 reduces or prevents IL-3 and/or GM-CSF-induced STAT-5 signaling.

In one example, the compound that binds to CD131 reduces or prevents IL-3-induced STAT-5 signaling with an IC₅₀ of about 20 nM or less. In one example, the pStat-5 IC₅₀ IL-3 is about 10 nM or less, or about 9 nM or less, or about 8 nM or less. In one example, the pStat-5 IC₅₀ IL-3 is about 7.5 nM or less, for example 7.3 nM.

In one example, the compound that binds to CD131 reduces or prevents GM-CSF-induced STAT-5 signaling with an IC₅₀ of about 60 nM or less. In one example, the pStat-5 IC₅₀ GM-CSF is about 50 nM or less, or about 45 nM or less or about 40 nM or less. In one example, the compound that binds to CD131 reduces or prevents GM-CSF-induced STAT-5 signaling with an IC₅₀ of about 40 nM.

For example, the compound can be contacted to a cell (e.g., a TF-1 cell) comprising a beta-lactamase reporter gene under control of the interferon regulatory factor 1 (irf1) response element in the presence of IL-3 and/or GM-CSF. Cells are also contacted with a suitable substrate (e.g., a negatively charged fluorescent beta-lactamase substrate, such as CCF2 or CCF4) and the change in signal fluorescence) determined. A reduced change in signal in a positive control (i.e., cells contacted with IL-3 and/or GM-CSF in the absence of the protein or antibody) indicates that the compound reduces or prevents IL-3 and/or GM-CSF-induced STAT-5 signaling.

In one example, the compound that binds to CD131 has one or more of the following activities:
(i) reduces or inhibits activation of isolated human neutrophils by GM-CSF as determined by reducing or inhibiting GM-CSF-induced increase in neutrophil cell size;
(ii) reduces or inhibits IL-3-induced IL-8 secretion by human basophils;
(iii) reduces or prevents IL-3-mediated survival or plasmacytoid dendritic cells (pDCs);
(iv) reduces or prevents activation of human peripheral blood eosinophils by IL-5 as determined by assessing change in forward scatter assessed by flow cytometry;
(v) reduces or prevents survival of human peripheral blood eosinophils in the presence of IL-5 and/or GM-CSF and/or IL-3;
(vi) reduces or prevents IL-3-induced tumor necrosis factor (TNF) α release from human mast cells;
(vii) reduces or prevents IL-3-induced IL-13 release from human mast cells;
(viii) reduces or prevents potentiation of IgE-mediated IL-8 release from human mast cells by IL-3 and/or IL-5 and/or GM-CSF;
(ix) reduces or prevents formation of colony forming units-granulocytes-macrophages (CFU-GM) by CD34+ human bone marrow cells cultured in the presence of stem cell factor (SCF), GM-CSF, IL-3 and IL-5;
(x) reduces the size or weight of polyps in a mouse xenograft model of human nasal polyposis; and/or
(xi) reduces the number of B cells in a polyp in a mouse xenograft model of human nasal polyposis.

In one example, the compound that binds to CD131 does not substantially or significantly inhibit proliferation of TF-1 cells in response to one or more of erythropoietin, IL-6, IL-4 or stein cell factor. Methods for determining the ability of the compound that binds to CD131 to inhibit proliferation of TF-1 cells in respect to a cytokine or growth factor are described herein and are readily adaptable to the present example of the disclosure.

In sonic examples, a compound useful in the present disclosure is a protein comprising an antigen binding site, for example a protein comprising an antigen binding site of an antibody or a single domain antibody. For example, the compound is an antibody or an antigen binding domain thereof.

In some examples, the compound that binds to CD131 is a protein comprising an antigen binding site that binds to CD131. In some examples, the antigen binding site is an antigen binding site of an antibody or a single domain antibody. In sonic examples, the antigen binding site comprises one or more CDRs.

Reference herein to a compound or protein or antibody that “binds to” CD131 provides literal support for a compound or protein or antibody that “binds specifically to” or “specifically binds to” CD131.

In one example, the K_{D} of the protein for a polypeptide comprising a sequence set forth in SEQ ID NO: 5 is about 10 nM or less, when the polypeptide is immobilized on a solid surface and the K_{D} is determined by surface plasmon resonance.

In one example, the K_{D} is 10 nM or less, for example, 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less. In one example, the K_{D} is 0.5 nM or less. In one example, the K_{D} is nM or less. In one example, the K_{D} is 0.3 nM or less. In one example, the K_{D} is 0.2 nM or less.

In one example, the protein comprising an antigen binding site binds to a cell expressing CD131 (e.g., a neutrophil or an eosinophil or a TF-1 cell) with a K_{D} of about 10 nM or less, e.g., using a competition assay using labeled and unlabeled protein or antibody. In one example, the K_{D} is 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less.

In one example, the K_{D} is about 300 nM or less for a neutrophil.

In one example, the K_{D} is about 700 nM or less for an eosinophil.

In one example, the K_{D} is about 400 nM or less for a TF-1 cell.

In one example, the protein comprising an antigen binding site is a protein comprising one or more antibody variable regions. In one example, the protein comprises a heavy chain variable region (V_{H}). In one example, the protein comprises a light chain variable region (V_{L}). In one example, the protein comprises a V_{H} and a V_{L}. In some examples, the V_{H} and a V_{L} are in the same polypeptide chain. In other examples, the V_{H} and a V_{L} are in separate polypeptide chains.

In some examples, the protein is a single domain antibody (sdAb).

In some examples, the protein comprises a Fv.

In some examples, the protein comprises:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

In some examples, the protein is selected from the group consisting of;

(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fe or a heavy chain constant domain (C_{H}) 2 and/or C_{H}3;
(x) one of (i) to (viii) linked to albumin, functional fragments or variants thereof or a protein (e.g., antibody or antigen binding fragment thereof) that hinds to albumin; or
(xi) an antibody.

In one example, the protein comprises an Fc region.

In one example, the protein comprises one or more amino acid substitutions that increase the half-life of the protein. In one example, the antibody comprises a Fc region comprising one or more amino acid substitutions that increase the affinity of the Fc region for the neonatal Fc receptor (FcRn).

In one example, the protein is an antibody, for example, a monoclonal antibody.

In one example, the antibody is a naked antibody.

In one example, the protein (or antibody) is chimeric, de-immunized, humanized, human or primatized.

In one example, the protein or antibody is human.

Exemplary antibodies include 9A2-VR24.29 (also, referred to as “CSL311”) described in WO 2017/088028 and BION-1 described in Sun et al. (1999) Blood 94:1943-1951.

In one example, the protein comprises a human constant region, e.g., an IgG constant region, such as an IgG1, IgG2, IgG3 or IgG4 constant region or mixtures thereof. In the case of a protein comprising a V_{H} and a V_{L}, the V_{H} can be linked to a heavy chain constant region and the V_{L} can be linked to a light chain constant region.

The C-terminal lysine of the heavy chain constant region of a whole antibody (or a protein comprising a constant region or a C_{H}3) may be removed, for example, during production or purification of the protein or antibody, or by recombinantly engineering the nucleic acid encoding the heavy chain. Accordingly, whole antibodies (or CD131-binding compounds) may comprise populations with all C-terminal lysine residues removed, populations with no C-terminal lysine residues removed, and/or populations having a mixture of protein with and without the C-terminal lysine residue. In some examples, the populations may additionally comprise protein in which the C-terminal lysine residue is removed in one of the heavy chain constant regions. Similarly, a composition of whole antibodies may comprise the same or a similar mix of antibody populations with or without the C-terminal lysine residue.

In one example, the protein is within a composition. For example, the composition comprises a protein comprising an antigen binding site or an antibody as described herein. In one example, the composition additionally comprises one or more variants of the protein or antibody. For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Dearnidated variants of encoded glutamine residues may result in glutamic acid. Compositions comprising a heterogeneous mixture of such sequences and variants are intended to be included when reference is made to a particular amino acid sequence.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, the heavy chain constant region comprises a sequence set forth in SEQ ID NO: 16. In one example, the protein, or a composition comprising the protein, comprises a heavy chain constant region, including a stabilized heavy chain constant region, comprising a mixture of sequences fully or partially with or without the C-terminal lysine residue.

In some examples, the protein comprises an antibody variable region that binds to CD131 and competitively inhibits the binding of antibody 9A2-VR24.29 comprising a V_{H} comprising a sequence set forth in SEQ ID NO: 6 and a V_{L} comprising a sequence set forth in SEQ ID NO: 7 to CD131.

In some examples, the protein comprises an antibody variable region that binds to CD131 and competitively inhibits the binding of antibody 9A2-VR24.29 comprising a V_{H} comprising a sequence set forth in SEQ ID NO: 6 and a V_{L} comprising a sequence set forth in SEQ ID NO: 18 to CD131.

In some examples, the protein hinds to the same or an overlapping epitope in CD131 as antibody 9A2-VR24.29 comprising a V_{H} comprising a sequence set forth in SEQ ID NO: 6 and a V_{L} comprising a sequence set forth in SEQ ID NO: 7 to CD131.

In some examples, the protein binds to the same or an overlapping epitope in CD131 as antibody 9A2-VR24.29 comprising a V_{H} comprising a sequence set forth in SEQ ID NO: 6 and a V_{L} comprising a sequence set forth in SEQ ID NO: 18 to CD131.

In some examples, the antigen binding site binds to an epitope within Site 2 of CD131. In this regard, the skilled artisan will be aware that Site 2 of CD131 is made up of residues from two CD131 polypeptides that form a dimer, e.g., Site 2 comprises residues within loops A-B and E-F of domain 1 of one CD131 polypeptide and residues within loops B-C and F-G of another CD131 polypeptide.

In some examples, the antigen binding site binds to an epitope formed upon dimerization of two CD131 polypeptides.

In some examples, the antigen binding site binds to residues within domain 1 of a CD131 polypeptide and residues within domain 4 of another CD131 polypeptide. In one example, the residues within domain 1 of CD131 comprise residues in the region of 101-107 of SEQ ID NO: 1 and/or the residues within domain 4 of CD131 comprise residues in the region of 364-367 of SEQ ID NO: 1.

In some examples, the protein binds to an epitope comprising
a) amino acids in one CD131 polypeptide chain corresponding to one or more or all of positions 39, 101, 102, 104, 105, 106, and 107 of SEQ ID NO: 1, and
b) amino acids in another CD131 polypeptide chain corresponding to one or more or all of positions 364, 365, 366, 367, 420, and 421 of SEQ ID NO: 1.

In some examples, the protein comprises an antibody variable region comprising a V_{H} comprising three CDRs of a V_{H} comprising an amino acid sequence set forth in SEQ ID NO: 6 and a V_{L} comprising three CDRs of a V_{L} comprising an amino acid sequence set forth in SEQ ID NO: 7.

In some examples, the protein comprises an antibody variable region comprising a V_{H} comprising three CDRs of a V_{H} comprising an amino acid sequence set forth in SEQ ID NO: 6 and a V_{L} comprising three CDRs of a V_{L} comprising an amino acid sequence set forth in SEQ ID NO: 18.

In some examples, the protein comprises
a) a V_{H} comprising a HCDR1 comprising or consisting of an amino acid sequence set forth in SEQ ID NO: 8 or a sequence having no more than one or two or three amino acid substitutions relative to SEQ ID NO: 8, a HCDR2 comprising or consisting of an amino acid sequence set forth in SEQ ID NO: 9 or a sequence having no more than one or two or three amino acid substitutions relative to SEQ ID NO: 9, and a HCDR3 comprising or consisting of an amino acid sequence set forth in SEQ ID NO: 10 or a sequence having no more than one or two or three amino acid substitutions relative to SEQ ID NO: 10; and
b) a V_{L} comprising a LCDR1 comprising or consisting of an amino acid sequence set forth in SEQ ID NO: 11 or a sequence having no more than one or two or three amino acid substitutions relative to SEQ ID NO: 11, a LCDR2 comprising or consisting of an amino acid sequence set forth in SEQ ID NO: 1:2 or a sequence having no more than one or two or three amino acid substitutions relative to SEQ ID NO: 12, and a LCDR3 comprising or consisting of an amino acid sequence set forth in SEQ ID NO: 13 or a sequence having no more than one or two or three amino acid substitutions relative to SEQ ID NO: 13.

In some examples, the protein comprises
a) a V_{H} comprising a HCDR1 comprising or consisting of an amino acid sequence set forth in SEQ ID NO: 8, a HCDR2 comprising or consisting of an amino acid sequence set forth in SEQ ID NO: 9, and a HCDR3 comprising or consisting of an amino acid sequence set forth in SEQ ID NO: 10; and
b) a V_{L} comprising a LCDR1 comprising or consisting of an amino acid sequence set forth in SEQ ID NO: H, a LCDR2 comprising or consisting of an amino acid sequence set forth in SEQ ID NO: 12, and a LCDR3 comprising or consisting of an amino acid sequence set forth in SEQ 11) NO: 13.

In some examples, the protein comprises a V_{H} comprising an amino acid sequence having at least 70%, at least 80%, at least 90%, or at least 95% sequence identity to SEQ ID NO: 6 and a V_{L} comprising an amino acid sequence having at least 70%, at least 80%, at least 90%, or at least 95% sequence identity to SEQ ID NO: 7.

In some examples, the protein comprises a V_{H} comprising an amino acid sequence having at least 70%, at least 80%, at least 90%, or at least 95% sequence identity to SEQ ID NO: 6 and a V_{L} comprising an amino acid sequence having at least 70%, at least 80%, at least 90%, or at least 95% sequence identity to SEQ ID NO: 18.

In some examples, the protein comprises a V_{H} comprising an amino acid sequence set forth in SEQ ID NO: 6 and a V_{L} comprising an amino acid sequence set forth in SEQ ID NO: 7.

In some examples, the protein comprises a V_{H} comprising an amino acid sequence set forth in SEQ ID NO: 6 and a V_{L} comprising an amino acid sequence set forth in SEQ ID NO: 18.

In some examples, the protein comprises a heavy chain comprising an amino acid sequence having at least 70%, at least 80%, at least 90%, or at least 95% sequence identity to SEQ ID NO: 14 and a light chain comprising an amino acid sequence having at least 70%, at least 80%, at least 90%, or at least 95% sequence identity to SEQ ID NO: 15.

In some examples, the protein comprises a heavy chain comprising an amino acid sequence set forth in SEQ ID NO: 14 and a light chain comprising an amino acid sequence set forth in SEQ ID NO: 15.

In one example, the compound that hinds to CD131 is administered in combination with another therapy.

In one example, the other therapy comprises administration of an anti-inflammatory compound. In one example, the other therapy comprises administration of an immunomodulator or an immunosuppressant.

In some examples, the other therapy comprises administration of a protein comprising an antigen binding site. In some examples, the protein comprising an antigen binding site is an antibody.

In some examples, the compound that binds to CD131 is administered in combination with a cell. In some examples, the cell is a stein cell, such as a mesenchymal stem cell.

In some examples, the compound that hinds to CD131 is administered in combination with a gene therapy.

In one example, the compound that binds to CD131 is administered simultaneously with the other therapy. In one example, the compound that binds to CD131 is administered before the other therapy. In one example, the compound that binds to CD131 is administered after the other therapy.

In some examples, the compound that binds to CD131 is administered in combination with a standard of care therapy. The standard of care therapy may be a standard of care therapy for the underlying cause of ARDS, or it may he a standard of care therapy for ARDS itself.

In some examples, the standard of care therapy comprises one or more or all of the following:
a) prone positioning;
b) fluid management;
c) administration of nitric oxide;
d) administration of a neuromuscular blocking agent;
e) artificial ventilation;
f) extracorporeal membrane oxygenation; and
g) administration of an antiviral agent or antibiotic.

In one example, the standard of care therapy comprises administration of an anti-viral. In one example, the standard of care therapy comprises administration of remdesivir. In one example, the standard of care therapy comprises administration of one or more of the following:
a) hydroxychloroquine;
b) chloroquine;
c) lopinavir;
d) ritonavir;
e) azithromycin;
f) interferon beta;
g) anakinra;
h) tocilizumab;
i) sarilumab;
j) dexamethasone;
k) aspirin;
l) losartan;
m) simvastatin; and
n) baricitinib.

In one example, the subject is a human. In one example, the subject is an adult, for example over 18 years of age. In one example, the subject is a child, for example less than 18 years of age. In one example, the subject is between 18 and 90 years of age. In one example, the subject is between 50 and 80 years of age.

In one example, the subject does not have chronic obstructive pulmonary disease (COPD). In one example, the subject does not have asthma.

### KEY TO SEQUENCE LISTING

SEQ ID NO 1: amino acid sequence of Homo sapiens CD131
SEQ ID NO 2: amino acid sequence of Homo sapiens IL-3-receptor α
SEQ ID NO 3: amino acid sequence of Homo sapiens GM-CSF receptor
SEQ ID NO 4: amino acid sequence of Homo sapiens IL-5 receptor
SEQ ID NO 5: amino acid sequence of soluble Homo sapiens CD131 comprising a C-terminal 6×His tag
SEQ ID NO 6: amino acid sequence of V_{H} of 9A2-VR24.29
SEQ ID NO 7: amino acid sequence of V_{L} of 9A2-VR24.29
SEQ ID NO 8: amino acid sequence of HCDR1 of 9A2-VR24.29
SEQ ID NO 9: amino acid sequence of HCDR2 of 9A2-VR24.29
SEQ ID NO 10: amino acid sequence of HCDR3 of 9A2-VR24.29
SEQ ID NO 11: amino acid sequence of LCDR1 of 9A2-VR24.29
SEQ ID NO 12: amino acid sequence of LCDR2 of 9A2-VR24.29
SEQ ID NO 13: amino acid sequence of LCDR3 of 9A2-VR24.29
SEQ ID NO 14: amino acid sequence of heavy chain of 9A2-VR24.29
SEQ ID NO 15: amino acid sequence of light chain of 9A2-VR24.29
SEQ ID NO 16: amino acid sequence of stabilized IgG4 heavy chain constant region
SEQ ID NO 17: amino acid sequence of kappa light chain constant region
SEQ ID NO 18: amino acid sequence of V_{L} of 9A2-VR24.29

### BRIEF DESCRIPTION OF THE DRAWINGS

FIG. 1 is a series of graphical representations of (A) cell numbers and (B) differential cells per ml (means +/− SEM) in stacked columns for lymphocytes, neutrophils and macrophages in the brocho-alveolar lavage fluid (BALE) of mice prophylactically administered with chimeric monoclonal antibody. Mice were administered with 50 mg/kg of chimeric monoclonal antibody (chimeric mAb) intravenously or isotype control (BM4) 24 h prior to intubation with 3 μg LPS or PBS described in Example 1. BALF of mice were collected 24 h after intubation for analysis. Significant difference in cell numbers between the chimeric mAb and BM4 groups were observed (ordinary one-way ANOVA Dunnett's multiple comparison test) (P=0.0004); n=3 (PBS), 6 (BM4_LPS) and 6 (chimeric mAb_LPS).

FIG. 2 is a series of graphical representations of (A) cell numbers and (B) differential cells per ml (means +/− SEM) in stacked columns for lymphocytes, neutrophils and macrophages in the brocho-alveolar lavage fluid (BALF) of mice therapeutically administered with chimeric monoclonal antibody described in Example 1. Mice were administered with 50 mg/kg of chimeric monoclonal antibody (chimeric mAb) intravenously or isotype control (BM4) 6 h after intubation with 3 μg LPS or PBS. BALE of mice were collected 24 h after intubation for analysis. Significant difference in cell numbers between the chimeric mAb and BM4 groups were observed (ordinary one-way ANOVA Dunnett's multiple comparison test) (P=0.0013); n=3 (PBS), 5 (BM4_LPS) and 6 (chimeric mAb_LPS).

FIG. 3 is a series of graphical representations showing inhibition of (A) GM-CSF and (B) IL-3 induced proliferation of FDCP1 cells by chimeric monoclonal antibody (chimeric mAb) compared to isotype control monoclonal. antibody (isotype control mAb) described in Example 2.

FIG. 4 is a series of graphical representations showing lung leukocyte numbers and markers of lung injury in mouse model of ARDS described in Example 3. Graph of (A) percentage body weight loss of hβcTg mice administered with LPS-ISO or CSL311 (B) blood granulocytes numbers (C) blood monocytes numbers and (D) brocho-alveolar lavage fluid (BALF) neutrophils numbers of mice administered with LPS-ISO or CSL311. (E) Immunohistochemically stained (H&E) section of lung from hβcTg mice administered with LPS-ISO or CSL311. Graphical representation showing (F) lung injury score based on H&E staining of lung sections (G) lung MPO activity and (H) BALF protein of hβcTg mice administered with LPS-ISO or CSL311. N=6-7,*<0.05, ANOVA vs. LPS-ISO groups.

FIG. 5 is a series of graphical representations showing netosis in mouse model of ARDS described in Example 3. (A) Immunohistochemical staining of lung sections of hβcTg mice administered with LPS-ISO or CSL311. MPO (green) and histone H3 (red) identified regions of netosis based on co-localisation of MPO and histone H3. Graphical representations quantifying (B) MPO activity (C) dsDNA (D) MPO activity (E) dsDNA in BALF of hβcTg mice administered with LPS-ISO or CSL311. N=6-7,*<0.05, ANOVA vs. LPS-ISO groups.

FIG. 6 is a graph showing percentage of blood oxygenation in mouse model of ARDS described in Example 3. Blood oxygenation of hβcTg mice administered with PBS (and no LPS), LPS-ISO or LPS-CSL311 was determined. Data are expressed as % oxygen saturation +/− SEM and statistical analysis, comparing the LPS-ISO and LPS-CSL311 groups, was done using a 2-way ANOVA with Bonferroni Post-test for multiple comparisons. *** indicates p<0.001; **indicates p<0.01; *indicates p<0.05. n=5 mice per group.

FIG. 7 is a series of graphical representations showing that βc cytokines and βc receptor are overexpressed in IAV infected hβcTg mice. hβcTg mice were infected with IAV (10⁴ PFU, HKx31 strain) and culled at day 3 and day 6 post infection. Gene expression for: βc cytokines—(A) Gm-csf, (B) Il3, (C) Il5; and βc receptor—(D) CSF2RB were analyzed in lung tissue by RTqPCR. n=6; data are Mean±SE; *p<**p<0.05, **p<0.01, ***p<0.001 one-way ANOVA.

FIG. 8 is a series of graphical representations showing that CSL311 reduces lung inflammation and injury without compromising viral clearance in IAV-infected hβcTg mice. CSL311 or isotype control (ISO) was administered to IAV-infected hβcTg mice at day 4 post infection. At day 7, IAV induced (A) body weight loss, which was not significantly improved by CSL311 treatment. (B) Lung viral load was measured by RTqPCR on viral PA gene and no difference was detected between CSL311 and ISO treated mice infected with IAV. Elevated levels of (C) blood monocytes, (D) blood neutrophils and (E) blood hemoglobin (HGB) induced by IAV infection were significantly reduced by CSL311. (F) BAL neutrophils and (G) BAL macrophages were also reduced with CSL311 treatment. n=6; data are Mean±SE except viral loads which are Median±interquartile ranges; * p<0.05, ** p<0.01, *** p<0.001 one-way ANOVA except viral loads which are Mann-Whitney U test.

FIG. 9 is a series of graphical representations showing that CSL311 blocks lung myeloid cells and preserves NK cell and T cells in IAV-infected hβcTg mice. Flow cytometry analysis utilizing a (A) gating strategy for myeloid cells revealed that lung (B) neutrophils, (C) alveolar macrophages (AMs), (D) exudative macrophages (EMs), (E) monocytes and (F) eosinophils were highly increased in hβcTg mice at day 6 post IAV infection, and significantly reduced with CSL311 treatment. Flow cytometry analysis utilizing a (G) gating strategy for delineating lymphoid cells demonstrated that lung (H) NK cells, (I) NKT cells and (J) regulatory T cells (Tregs) were increased following IAV infection and were not affected by CSL311 treatment. (K) Lung CD4 cells and (L) CD8 cell numbers in the lungs did not differ across all groups; n=6; data are Mean±SE;* p<0.05, ** p<0.01, *** p<0.001 one-way ANOVA.

FIG. 10 is a series of graphical representations showing that CSL311 attenuates lung cytokine storm and preserves interferon expression in IAV-infected hβcTg mice. Expression of the neutrophil chemokine (A) Cxcl1, monocyte/macrophage chemokine (B) Ccl2, eosinophil chemokine (C) Ccl24, T cell/NK cell chemokine (D) Cxcl10 were detected by RTqPCR in the lungs of IAV-infected mice, where CSL311 significantly lowered Ccl2 and Ccl24 levels. Expression of the pro-inflammatory genes (E) Il1a and (F) Il6 were also increased in IAV-infected mice and CSL31 I treatment significantly reduced Il1a but not Il6 expression. (G) Type I interferon (Ifnb), (H) type II interferon (Ifng) and (I) type III interferon (Ifnl2/3) were also markedly induced in the lungs of IAV-infected mice in a manner that was not significantly altered by CSL311 treatment; n=6; * p<0.05, data are Mean±SE; ** p<0.01, *** p<0.001 one-way ANOVA.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of flatter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure. Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A

Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Names (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901 -917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

For the purposes of nomenclature only and not limitation an exemplary sequence of a human CD131 (pre-CD131) is set out in NCBI Reference Sequence: NP_000386.1 and NCBI Genbank Accession Number P32927 (and set out in SEQ ID NO: 1). A sequence of a mature human CD131 lacks amino acids 1 to 16 of SEQ ID NO: 1. Positions of amino acids are often referred to herein by reference to pre-CD131. The positions in mature CD131 is readily determined by accounting for the signal sequence (amino acids 1-16 in the case of SEQ ID NO: 1). The sequence of CD131 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human CD131 may be abbreviated to hCD131. Reference to soluble CD131 refers to polypeptides comprising the extracellular region of CD131, e.g., amino acids 17 to 438 of SEQ ID NO: 1.

Reference herein to CD131 includes native forms of CD131 and mutant forms thereof retaining an ability to bind to CD131 (e.g., hCD131) and induce signaling. CD131 is also known as “CSF2RB” and “cytokine receptor common subunit beta” and “β (beta) common receptor” (abbreviated as “βCR” or “βc”).

A “compound”, as contemplated by the present disclosure, can take any of a variety of forms including natural compounds, chemical small molecule compounds or biological compounds or macromolecules. Exemplary compounds include an antibody or a protein comprising an antigen binding fragment of an antibody, a nucleic acid, a polypeptide, a peptide, and a small molecule.

As used herein, the term “disease” or “condition” refers to a disruption of or interference with normal function, and is not to be limited to any specific condition, disease or disorder.

As used herein, the terms “treating”, “treat” or “treatment” include administering a compound described herein to reduce, prevent, or eliminate at least one symptom of a specified disease or condition.

As used herein, the terms “preventing”, “prevent” or “prevention” include administering a compound described herein to thereby stop or hinder the development of at least one symptom of a condition, e.g., before that symptom is fully developed in the subject.

As used herein, the term “subject” shall be taken to mean any animal including humans, for example a mammal. Exemplary subjects include but are not limited to humans and non-human primates. In one example, the subject is a human.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. In some examples, the protein is a fusion protein. As used herein, a “fusion protein” is a protein comprising at least two domains that have been joined so that they are translated as a single unit, producing a single protein.

The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some examples, an antigen binding site is or comprises a V_{H} or a V_{L} or a Fv. In sonic examples, the antigen binding site comprises one or more CDRs of an antibody.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is a, α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fe region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not hind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to CD131 with materially greater affinity (e.g., 20 fold or 40 fold or 60 fold or 80 fold to 100 fold or 150 fold or 200 fold) than it does to other cytokine receptors or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

A protein or antibody may he considered to “preferentially bind” to a polypeptide if it binds that polypeptide with a dissociation constant (K_{D}) that is less than the protein's or antibody's K_{D} for another polypeptide. In one example, a protein or antibody is considered to preferentially bind to a polypeptide if it binds the polypeptide with an affinity (i.e., K_{D}) that is at least about 20 fold or 40 fold or 60 fold or 80 fold or 100 fold or 120 fold or 140 fold or 160 fold more than the protein's or antibody's K_{D} for another polypeptide.

For the purposes of clarification and as will be apparent to the skilled artisan based on the exemplified subject matter herein, reference to “affinity” in this specification is a reference to K_{D} of a protein or antibody.

For the purposes of clarification and as will be apparent to the skilled artisan based on the description herein, reference to “a K_{D} of X nM or less” will be understood to mean that the numerical value of the K_{D} is equal to X nM or is lower in numerical value. As a skilled person would understand a lower numerical value of a K_{D} corresponds to a higher (i.e., stronger) affinity, i.e., an affinity of 2 nM is stronger than an affinity of 3 nM.

An “IC₅₀ of at least about” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

As used herein, the term “epitope” (syn. “antigenic determinant”) shall be understood to mean a region of CD131 to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when CD131 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a protein of the disclosure (or an antigen binding site thereof) reduces or prevents binding of a recited antibody or protein to CD131. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to CD131 either in the presence or absence of the protein. If less antibody binds in the presence of the protein than in the absence of the protein, the protein is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 20 amino acids.

### Acute Respiratory Distress Syndrome (ARDS)

The present disclosure provides, for example, a method for treating or preventing acute respiratory distress syndrome (ARDS) in a subject.

ARDS is a life-threatening condition characterized by bilateral pulmonary infiltrates, severe hypoxemia, and non-cardiogenic pulmonary edema. ARDS results in severe lung damage and an attributable mortality rate of 30-50%. As yet, there is no effective pharmacological therapy. Infectious etiologies, such as sepsis and pneumonia (including influenza and coronavirus infection), are leading causes of ARDS. Therefore, the treatment of these underlying diseases or disorders (e.g., the infection) is also contemplated in combination with the methods of the disclosure.

Histologically, ARDS in humans is characterized by a severe acute inflammatory response in the lungs and neutrophilic alveolitis. Inflammatory stimuli from microbial pathogens, such as endotoxin (lipopolysaccharide, LPS), are well recognized for their ability to induce pulmonary inflammation, and experimental administration of LPS, both systemically and intratracheally, has been used to induce pulmonary inflammation in animal models of ARDS, as described herein. LPS acts via Toll-like receptor 4 (TLR4), to increase the expression of inflammatory cytokines and chemokines, and upregulate leukocyte adhesion molecules, resulting in endothelial cell activation.

The physiological hallmark of ARDS is disruption of the alveolar-capillary membrane barrier (i.e., pulmonary vascular leak), leading to development of non-cardiogenic pulmonary edema in which a proteinaceous exudate floods the alveolar spaces, Impairs gas exchange, and precipitates respiratory failure. Both alveolar epithelial and endothelial cell injury and/or death have been implicated in the pathogenesis of ARDS. ARDS continues to he a significant contributor to prolonged mechanical ventilation in the intensive care unit (ICU), and ARDS-associated mortality remains high at 30-50% despite optimal ICU supportive care.

ARDS was defined by a panel of experts in 2012 (an initiative of the European Society of Intensive Care Medicine endorsed by the American Thoracic Society and the Society of Critical Care Medicine) as the Berlin Definition. Presently there are three stages: mild, moderate, and severe with an associated increased mortality (27%; 95% CI, 24%-30%; 32%; 95% CI, 29%-34%; and 45%; 95% CI, 42%-48%, respectively ; P<0.001) and increased median duration of mechanical ventilation in survivors (5 days; interquartile [IQR], 2-11; 7 days; IQR, 4-14; and 9 days; IQR, 5-17, respectively; P<0.001). The definition was empirically evaluated using patient-level meta-analysis of 4188 patients with ARDS from 4 multicenter clinical data sets and 269 patients with ARDS from 3 single-center data sets containing physiologic information.

According to the Berlin definition, ARDS is defined by
(1) presentation within 1 week of clinical insult or onset of respiratory symptoms;
(2) acute hypoxemic respiratory failure, as determined by a PaO₂/FiO₂ ratio of 300 mmHg or less on at least 5 cm of continuous positive airway pressure (CPAP) or positive end expiratory pressure (PEEP), where PaO₂ is the partial pressure of oxygen in arterial blood and the FiO₂ is the fraction of inspired oxygen;
(3) bilateral opacities on lung radiographs not fully explained by effusions, consolidation, or atelectasis; and
(4) edema/respiratory failure not fully explained by cardiac failure or fluid overload.

In an example of the methods of the present disclosure, the subject satisfies the above Berlin criteria for ARDS. In other examples, the subject may not yet satisfy the Berlin criteria for ARDS but is identified as at risk of developing ARDS. Such subjects can be administered the compound that binds to CD131 to prevent onset of ARDS.

As used herein, the term “at risk” means that the subject has an increased chance of developing ARDS compared to a normal individual. Subjects can be identified as at risk of developing ARDS using any method known in the art. For example, the subject may be identified at risk of developing ARDS if that subject has a common underlying cause of ARDS (e.g., sepsis, pneumonia, trauma etc.) and has respiratory symptoms, for example, fast breathing and/or shortness of breath. Other methods suitable for identifying subjects at risk of developing ARDS include, for example, the methods described in WO2018/204509; Luo et al., 2017, J Thorac Dis 9, 3979-3995; de Haro et al., 2013, Annals of Intensive Care 3, 11; Iriyama et al., 2020, Journal of Intensive Care 8, 7; Gajic et al., 2011, Am J Respir Crit Care Med 183, 462-470; and Yadav et al., 2017, Am J Respir Crit Care Med 195, 725-736.

The severity of ARDS can be categorised as follows:
(1) Mild ARDS: PaO₂/FiO₂ of 200-300 mmHg;
(2) Moderate ARDS: PaO₂/FiO₂ of 100-200 mmHg; and
(3) Severe ARDS: PaO₂/FiO₂ of less than or equal to 100 mmHg.

In some examples of the methods of the disclosure, the ARDS is mild ARDS. In some examples, the ARDS is moderate ARDS. In some examples, the ARDS is severe ARDS.

The methods of the present disclosure are suited to all causes of ARDS. The most common causes of ARDS are sepsis, aspiration of harmful substances, pneumonia, severe trauma (bilateral lung contusion, fat embolism after long bone fracture, sepsis that develops several days after severe trauma or burns, and massive traumatic tissue injury), massive transfusion, transfusion related acute lung injury, lung and hematopoietic stem cell transplantation, other acute inflammatory diseases, drugs and alcohol, and genetic determinants such as mutations in the surfactant protein B (SP-B) gene.

More recently, ARDS has been shown to result from severe coronavirus disease 2019 (COVID-19), i.e., viral pneumonia from SARS-CoV-2 (severe acute respiratory syndrome coronavirus 2) infection. The methods of the present disclosure can be used to treat or prevent ARDS in a subject suffering from severe COVID-19. Thus, in some examples, the present disclosure provides a method of treating COVID-19 in a subject, the method comprising administering a compound that binds to CD131 and neutralizes signaling by IL-3, IL-5 and GM-CSF to the subject. Common symptoms of COVID-19 include fever, cough, fatigue, shortness of breath, and loss of smell and taste. While the majority of cases result in mild symptoms, some progress to ARDS. Other coronavirus infections in the past have caused SARS and MERS, which also can result in ARDS. SARS-CoV-2 infection can be confirmed, for example, by positive detection of viral RNA in nasopharyngeal secretions using a specific PCR test. COVID-19 illness can be confirmed by a consistent clinical history, epidemiological contact, and a positive SARS-CoV-2 test. ARDS associated with COVID-19 can be diagnosed when a subject with confirmed COVID-19 infection meets the Berlin ARDS diagnostic criteria described above.

### Antibodies

In one example, a compound as described herein according to any example is a protein comprising an antigen binding site of an antibody. In some examples, the compound is an antibody.

Methods for generating antibodies are known in the art and/or described in Harlow and Lane (editors) Antibodies: A Laboratory Manual, Cold Spring Harbor Laboratory, (1988). Generally, in such methods CD131 or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. Nos. 6,300,064 and/or 5,885,793. For example, the present inventors have isolated fully human antibodies from a phage display library.

An antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. Nos. 5,225,539, or 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

Exemplary human antibodies are described herein and include 9A2-VR24.29 (also, referred to as “CSL-311”) described in WO 2017/088028 and BION-1 described in Sun et al. (1999) Blood 94:1943-1951 and/or proteins comprising variable regions thereof or derivatives thereof. These human antibodies provide an advantage of reduced immunogenicity in a human compared to non-human antibodies.

In one example, the antibody is a multispecific antibody. For instance, the compound that binds to CD131 may be a protein comprising an antigen binding site that binds to CD131 and a further antigen binding site that hinds to a different antigen. Thus, in some examples, the antibody is a bispecific antibody.

### Antibody Binding Domain Containing Proteins

### Single-Domain Antibodies

In some examples, a compound of the disclosure is a protein that is or comprises a single-domain antibody (which is used interchangeably with the term “domain antibody” or “dAb” or “nanobody”). A single-domain antibody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). In certain examples, a single-domain antibody is a human single-domain antibody (Demands, Inc., Waltham, MA; see, e.g., U.S. Pat. No. 6,248,516).

In some examples, the single-domain antibody is a V_{H}H fragment. V_{H}H fragments consist of the variable domain (V_{H}) of camelid heavy-chain antibodies, described below.

In some examples, the single-domain antibody is a V_{NAR} fragment. V_{NAR} fragments consist of the variable domain (V_{H}) of heavy-chain antibodies from cartilaginous fish, described below.

### Diabodies, Triabodies, Tetrabodies

In some examples, a protein of the disclosure is or comprises a diabody, triabody, tetrabody or higher order protein complex such as those described in WO98/044001 and/or WO94/007921.

Single Chain (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### V-Like Proteins

An example of a compound of the disclosure is a T-cell receptor. T cell receptors have two V-domains that combine into a structure similar to the Fv module of an antibody. Novotny et al., Proc Natl Acad Sri USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can he engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002/088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two a-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### De-immunized Proteins

The present disclosure also contemplates a de-immunized antibody or protein. De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO2000/34317, WO2004/108158 and WO2004/064724.

Methods for introducing suitable mutations and expressing and assaying the resulting protein will he apparent to the skilled artisan based on the description herein.

### Mutations to Proteins

The present disclosure also contemplates mutant forms of a protein of the disclosure. For example, such a mutant protein comprises one or more conservative amino acid substitutions compared to a sequence set forth herein. In some examples, the protein comprises 30 or fewer or 20 or fewer or 10 or fewer, e.g., 9 or 8 or 7 or 6 or 5 or 4 or 3 or 2 conservative amino acid substitutions, A “conservative amino acid substitution” is one in which the amino acid residue is replaced with an amino acid residue having a similar side chain and/or hydropathicity and/or hydrophilicity.

In one example, a mutant protein has only, or not more than, one or two or three or four or five or six conservative amino acid changes when compared to a naturally occurring protein. Details of conservative amino acid changes are provided below. As the skilled person would be aware, e.g., from the disclosure herein, such minor changes can reasonably be predicted not to alter the activity of the protein.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

The present disclosure also contemplates non-conservative amino acid changes (e.g., substitutions) in a protein of the present disclosure, e.g., in a CDR, such as CDR3. In one example, the protein comprises fewer than 6 or 5 or 4 or 3 or 2 or 1 non-conservative amino acid substitutions, e.g., in a CDR3, such as in a CDR3.

The present disclosure also contemplates one or more insertions or deletions compared to a sequence set forth herein. In some examples, the protein comprises 10 or fewer, e.g., 9 or 8 or 7 or 6 or 5 or 4 or 3 or 2 insertions and/or deletions.

### Constant Regions

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fe region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fe region containing protein are known in the art and/or described herein.

In one example, the Fe region is an IgG4 Fe region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fe region comprising a substitution at one or more positions selected from the group consisting of 268, 309, 330 and 331. In another example, the Fc region is an IgG1 Fe region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al. J Immunol. 177: 1129-1138 2006; and/or Hezareh J Virol ;75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present disclosure also contemplates additional modifications to an antibody or protein of the disclosure.

For example, the antibody comprises one or more amino acid substitutions that increase the half-life of the protein. For example, the antibody comprises a Fc region comprising one or more amino acid substitutions that increase the affinity of the Fc region for the neonatal Fc receptor (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fe into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

The protein may he a fusion protein. Thus, in one example, the protein additionally comprises albumin, a functional fragment or variant thereof. In one example, the albumin, functional fragment or variant thereof is serum albumin, such as human serum albumin. In one example, the albumin, functional fragment or variant thereof, comprises one or more amino acid substitutions, deletions or insertions, e.g., no more than 5 or 4 or 3 or 2 or 1 substitutions. Amino acid substitutions suitable for use in the present disclosure will be apparent to the skilled person and include naturally-occurring substitutions and engineered substitutions such as those described, for example, in WO2011/051489, WO2014/072481, WO2011/103076, WO2012/112188, WO2013/075066, WO2015/063611 and WO2014/179657.

In one example, the protein of the disclosure additionally comprises a soluble complement receptor or functional fragment or variant thereof. In one example, the protein additionally comprises a complement inhibitor.

### Protein Production

In one example, a protein described herein according to any example is produced by culturing a hybridoma under conditions sufficient to produce the protein, e.g., as described herein and/or as is known in the art.

### Recombinant Expression

In another example, a protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1α-promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can he purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing.

These methods are known in the art and described, for example in WO1999/57134 or Ed Harlow and David Lane (editors) Antibodies: A Laboratory Manual, Cold Spring Harbor Laboratory, (1988).

The skilled artisan will also he aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that hinds to a tag is used in an affinity purification method.

### Nucleic Acid Compounds that Bind to CD131

In one example, the compound that binds to CD131 is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., CD131. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can he prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., hinds specifically to CD131) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or 5,475,096.

### Assaying Activity of a Compound

### Binding to CD131

Methods for assessing binding of a candidate compound to a protein (e.g., CD131) are known in the art, e.g., as described in Scopes (In: Protein purification: principles and practice, Third Edition, Springer Verlag, 1994). Such a method generally involves labeling the protein and contacting it with immobilized compound. Following washing to remove non-specific bound protein, the amount of label and, as a consequence, bound protein is detected. Of course, the protein can be immobilized and the compound labeled. Panning-type assays can also be used. Alternatively, or additionally, surface plasmon resonance assays can be used. The level of binding can also he conveniently determined using a biosensor.

Optionally, the dissociation constant (Kd) of a compound for CD131 or an epitope thereof is determined. The “Kd” or “Kd value” for a compound that binds to CD131 is in one example measured by a radiolabeled or fluorescently-labeled CD131 binding assay. This assay equilibrates the compound with a minimal concentration of labeled CD131 in the presence of a titration series of unlabeled CD131. Following washing to remove unbound CD131, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized CD131 or a region thereof.

### Epitope Mapping

In another example, the epitope bound by a protein described herein is mapped. Epitope mapping methods will he apparent to the skilled artisan. For example, a series of overlapping peptides spanning the CD131 sequence or a region thereof comprising an epitope of interest, e.g., peptides comprising 10-15 amino acids are produced. The protein is then contacted to each peptide and the peptide(s) to which it hinds determined. This permits determination of peptide(s) comprising the epitope to which the protein binds. If multiple non-contiguous peptides are bound by the protein, the protein may bind a conformational epitope.

Alternatively, or in addition, amino acid residues within CD131 are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding CD131 or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in CD131 or a region thereof to deutrons and binding the resulting protein to an immobilized protein of the present disclosure. The deutrons are then converted hack to hydrogen, the CD131 or region thereof isolated, digested with enzymes and analyzed, e.g., using mass spectrometry to identify those regions comprising deutrons, which would have been protected from conversion to hydrogen by the binding of a protein described herein.

Alternatively, the epitope to which the protein binds can be determined by X-ray crystallography. For example, a complex between the protein and CD131 is formed and then crystalized. The resulting crystals are then subjected to x-ray diffraction analysis to determine the atomic co-ordinates of the amino acids in the complex. The epitope comprises the amino acids in CD131 that are in contact with the protein, according to the atomic co-ordinates determined from the x-ray diffraction.

### Determining Competitive Binding

Assays for determining a protein that competitively inhibits binding of antibody 9A2-VR24.29 will be apparent to the skilled artisan. For example, 9A2-VR24.29 is conjugated to a detectable label, e.g., a fluorescent label or a radioactive label. The labeled antibody and the test protein are then mixed and contacted with CD131 or a region thereof (e.g., a polypeptide comprising SEQ ID NO: 1 or 5) or a cell expressing same. The level of labeled 9A2-VR24.29 is then determined and compared to the level determined when the labeled antibody is contacted with the CD131, region or cells in the absence of the protein. If the level of labeled 9A2-VR24.29 is reduced in the presence of the test protein compared to the absence of the protein, the protein is considered to competitively inhibit binding of 9A2-VR24.29 to CD131.

Optionally, the test protein is conjugated to different label to 9A2-VR24.29. This alternate labeling permits detection of the level of binding of the test protein to CD131 or the region thereof or the cell.

In another example, the protein is permitted to bind to CD131 or a region thereof (e.g., a polypeptide comprising SEQ ID NO: 1 or 5) or a cell expressing same prior to contacting the CD131, region or cell with 9A2-VR24.29. A reduction in the amount of hound 9A2-VR24.29 in the presence of the protein compared to in the absence of the protein indicates that the protein competitively inhibits 9A2-VR24.29 binding to CD131. A reciprocal assay can also be performed using labeled protein and first allowing 9A2-VR24.29 to bind to CD131. In this case, a reduced amount of labeled protein bound to CD131 in the presence 9A2-VR24.29 compared to in the absence of 9A2-VR24.29 indicates that the protein competitively inhibits binding of 9A2-VR24.29 to CD131.

Any of the foregoing assays can be performed with a mutant form of CD131 and/or SEQ ID NO: 1 or 5 and/or a ligand binding region of CD131 to which 9A2-VR24.29 binds, e.g., as described herein.

### Determining Neutralization

In one example, the compound that binds to CD131 reduces or prevents binding of IL-3, IL-5 and/or GM-CSF to a receptor comprising CD131 (e.g., an IL-3R, an IL-5R and/or a GM-CSF-R, respectively). These assays can be performed as a competitive binding assay using labeled IL-3/Il-5/GM-CSF and/or labeled compound. For example, cells expressing the relevant receptor is contacted with IL-3/Il-5/GM-CSF in the presence or absence of a CD131-binding compound and the amount of bound label detected. A reduction in the amount of bound label in the presence of the CD131-binding compound compared to in the absence of the compound indicates that the compound reduces or prevents binding of IL-3/Il-5/GM-CSF to a receptor comprising CD131. By testing multiple concentrations of the compound an IC₅₀ is determined, i.e., a concentration of the compound that reduces the amount of 1L-3/Il-5/GM-CSF that binds to a receptor comprising CD131, or an EC₅₀ can be determined, i.e., a concentration of the protein that achieves 50% of the maximum inhibition of binding of IL-3/Il-5/GM-CSF to CD131 achieved by the compound.

In a further example, the CD131-binding compound reduces or prevents IL-3/Il-5/GM-CSF-mediated proliferation of leukemic cell line TF-1. For example, TF-1 cells are cultured without IL-3/Il-5/GM-CSF for a time sufficient for them to stop proliferating (e.g., 24-48 hours). Cells are then cultured in the presence of IL-3/Il-5/GM-CSF and various concentrations of the CD131-binding compound. Control cells are not contacted with the compound (positive control) or IL-3/Il-5/GM-CSF (negative control). Cell proliferation is then assessed using a standard technique, e.g., ³H-thymidine incorporation. A CD131-binding compound that reduces or prevents cell proliferation in the presence of IL-3 to a level less than the positive control is considered to neutralize IL-3 signaling. By testing multiple concentrations of the CD131-binding compound, an IC₅₀ is determined.

In another example, a CD131-binding compound inhibits or prevents STAT-5 activation. For example, cells (e.g., TF-1 cells) comprising a beta-lactamase reporter gene under control of the interferon regulatory factor 1 (irf1) response element in the presence of IL-3 and/or GM-CSF. Suitable cells are available from Life Technologies Corporation. Cells are also contacted with a suitable substrate (e.g., a negatively charged fluorescent beta-lactamase substrate, such as CCF2 or CCF4) and the change in signal (e.g., fluorescence) determined. A reduced change in signal in a positive control (i.e., cells contacted with IL-3 and/or GM-CSF in the absence of the compound) indicates that the compound reduces or prevents IL-3 and/or GM-CSF-induced STAT-5 signaling.

In a further example, a CD131-binding compound of the disclosure affects an immune cell. For example, the CD131-binding compound reduces or inhibits activation of isolated human neutrophils by GM-CSF as determined by reducing or inhibiting GM-CSF-induced increase in neutrophil cell size. For example, neutrophils (e.g., about 1×10⁵ cells) are cultured in the presence of the CD-131-binding protein and GM-CSF for a suitable time (e.g., about 24 hours). Cells are then fixed (e.g., with formaldehyde) and analyzed for forward scatter using flow cytometry.

In one example, the CD131-binding compound reduces or inhibits IL-3-induced IL-8 secretion by human basophils. For example, basophils (e.g., about 1×10⁵ cells) are cultured in the presence of a CD131-binding compound and IL-3 for a suitable time (e.g.., 24 hours). IL-8 secretion is then assessed, e.g., using an ELISA, e.g., as is available from R&D Systems.

In a further example, the CD131-binding compound reduces or prevents IL-3-mediated survival or pDCs. For example, pDCs are cultured in the presence of a CD131-binding compound and IL-3 for a suitable time (e.g., 24 hours). Cell survival is then assessed, e.g., using a standard method, such as a ViaLight Plus Kit from Lonza.

In a further example, the CD131-binding compound reduces or prevents activation of human peripheral blood eosinophils by IL-5 as determined by assessing change in forward scatter assessed by flow cytometry. For example, eosinophils (e.g., about 1×10⁵ cells) are cultured in the presence of a CD131-binding compound and IL-5 for a suitable time (e.g., about 24 hours). Cells are then fixed in formaldehyde) and assessed for change in forward scatter, e.g., using flow cytometry.

In a further example, a CD131-binding compound of the disclosure reduces or prevents survival of human peripheral blood eosinophils in the presence of IL-5 and/or GM-CSF and/or IL-3. For example, eosinophils (e.g., about 1×10⁴ cells) are cultured in the presence of a CD131-binding compound and IL-5 and/or GM-CSF and/or IL-3 for a suitable time (e.g., about 5 days) and cell numbers assessed using a standard method (e.g., a ViaLight Plus Kit from Lonza).

In a still further example, a CD131-binding compound of the disclosure reduces or prevents IL-3-induced TNFα release from human mast cells. For example, human cultured mast cells (e.g., ten-week old peripheral blood-derived cells) are cultured in the presence of a CD131-binding compound and 11,3. Levels of TNFα secretion are then assessed by, e.g., ELISA.

In a further example, a CD131-binding compound of the disclosure reduces or prevents IL-3-induced IL-13 release from human mast cells. For example, human cultured mast cells (e.g., ten-week old peripheral blood-derived cells) are cultured in the presence of a CD131-binding compound and IL-3. Levels of IL-13 secretion are then assessed by, e.g., ELISA.

In a further example, a CD131-binding compound of the disclosure reduces or prevents potentiation of IgE-mediated IL-8 release from human mast cells by IL-3 and/or IL-5 and/or GM-CSF. For example, human cultured mast cells (e.g., ten-week old peripheral blood-derived cells) are cultured in the presence of a CD131-binding compound and IL-3/IL-5/GM-CSF (e.g., for about 48 hours). Cells are then cultured with IgE (e.g., human myeloma IgE) for a suitable time (e.g., about 24 hours) and IL-8 secretion assessed, e.g., by ELISA.

In a further example, a CD131-binding compound reduces or prevents formation of CFU-GM by CD34+ human bone marrow cells (or cord blood cells) cultured in the presence of SCF, GM-CSF, IL-3 and IL-5. For example, CD34+ cells (e.g., about 1×10³ cells) are cultured (e.g., on methylcellulose (such as 1% methylcellulose) supplemented with fetal calf serum, bovine serum albumin, SCF, GM-CSF, IL-3 and IL-5) and in the presence of a CD131-binding compound. Cells are cultured for a suitable time (e.g., about 16 days) and the number of colonies formed subsequently enumerated.

In a further example, a CD131-binding compound reduces survival of or induces death of immune cells (e.g., eosinophils) from sputum or nasal polyp tissue from a subject suffering from an inflammatory airway disease or nasal polyposis. For example, the immune cells are cultured in the presence of IL-3 and/or IL-5 and/or GM-CSF and the protein or antibody. Cell death is then assessed using standard methods, e.g., by detecting Annexin-V expression, e.g., using fluorescence activated cell sorting).

In another example, the CD131-binding compound reduces or prevents IL-3-mediated histamine release from basophils. For example, low density leukocytes comprising basophils are incubated with IgE, IL-3 and various concentrations of the antibody or antigen binding fragment. Control cells do not comprise immunoglobulin (positive control) or IL-3 (negative control). The level of released histamine is then assessed using a standard technique, e.g., RIA. A CD131-binding compound that reduces the level of histamine release to a level less than the positive control is considered to neutralize IL-3 signaling. In one example, the level of reduction is correlated with protein concentration. An exemplary method for assessing IL-3-mediated histamine release is described, for example, in Lopez et al., J. Cell. Physiol., 145: 69, 1990.

Another assay for assessing IL-3 signaling neutralization comprises determining whether or not the CD131-binding compound reduces or prevents IL-3-mediated effects on endothelial cells. For example, human umbilical vein endothelial cells (HUVECs) are cultured in the presence of IL-3 (optionally, with IFN-γ) and various concentrations of the CD131-binding compound. The amount of secreted IL-6 is then assessed, e.g., using an enzyme linked immunosorbent assay (ELISA). Control cultures do not comprise the CD131-binding compound (positive control) or IL-3 (negative control). A CD131-binding compound that reduces or prevents IL-6 production in the presence of IL-3 to a level less than the positive control is considered to neutralize IL-3 signaling.

Other methods for assessing neutralization of GM-CSF, IL-5 or IL-3 signaling are contemplated by the present disclosure.

### Determining Effector Function

As discussed herein, some CD131-binding compounds have reduced effector function or have effector function (or enhanced effector function). Methods for assessing ADCC activity are known in the art.

In one example, the level of ADCC activity is assessed using a ⁵¹Cr release assay, an europium release assay or a ³⁵S release assay. In each of these assays, cells expressing CD131 are cultured with one or more of the recited compounds for a time and under conditions sufficient for the compound to be taken up by the cell. In the case of a ³⁵S release assay, cells expressing CD131 can be cultured with ³⁵S-labeled methionine and/or cysteine for a time sufficient for the labeled amino acids to be incorporated into newly synthesized proteins. Cells are then cultured in the presence or absence of the CD131-binding compound and in the presence of immune effector cells, e.g., peripheral blood mononuclear cells (PBMC) and/or NK cells. The amount of ⁵¹Cr, europium and/or ³⁵S in cell culture medium is then detected, and little or no change in the presence of the CD131-binding compound compared to in the absence of the CD131-binding compound indicates that the protein has reduced effector function and an increased amount compared to in the absence of the CD131-binding compound (or increased compared to in the presence of the CD131-binding compound comprising an IgG1 Fc region) indicating effector function or enhanced effector function. Exemplary publications disclosing assays for assessing the level of ADCC induced by a protein include Hellstrom, et al. Proc. Nail Acad. Sci. USA 83:7059-7063, 1986 and Bruggemann, et al., J. Exp. Med. 66:1351-1361, 1987.

Other assays for assessing the level of ADCC induced by a protein include ACTI™ nonradioactive cytotoxicity assay for flow cytometry (CellTechnology, Inc. CA, USA) or CytoTox 96® non-radioactive cytotoxicity assay (Promega, WI, USA).

C1q binding assays may also be carried out to confirm that the CD131-binding compound is able to bind C1q and may induce CDC. To assess complement activation, a CDC assay may be performed (see, for example, Gazzano-Santoro et al, J. Immunol. Methods 202: 163, 1996).

### Determining Half Life

Some proteins encompassed by the present disclosure have an improved half-life, e.g., are modified to extend their half-life compared to proteins that are unmodified. Methods for determining a protein with an improved half-life will be apparent to the skilled person.

The half-life of a protein of the disclosure can be measured by in vivo pharmacokinetic studies, e.g., according to the method described by Kim et al, Eur J of Immunol 24:542, 1994. According to this method radiolabeled protein is injected intravenously into mice and its plasma concentration is periodically measured as a function of time, for example at 3 minutes to 72 hours after the injection. Alternatively, or additionally, other species can be used, e.g. cynomolgus monkeys and humans, and/or non-radiolabeled proteins can be injected and subsequently detected using an enzyme-linked immunosorbent assay (ELISA). The clearance curve thus obtained should be biphasic, that is, an alpha phase and beta phase. For the determination of the in vivo half-life of the protein, the clearance rate in beta-phase is calculated and compared with that of the wild type or unmodified protein.

The relative affinity of binding of a protein to the neonatal Fc receptor (FcRn) can also be indicative of its relative in vivo half-life (see for example, Kim et al., Eur J Immunol., 24:2429, 1994).

### Therapeutic Efficacy

The therapeutic efficacy of a compound that binds to CD131 can be assessed by comparing the degree of severity of the disease or symptoms in subjects administered with the compound relative to subjects not administered the compound. Alternatively, or additionally, therapeutic efficacy of candidate compounds can be assessed in an animal model.

Intratracheal lipopolysaccharide (LPS)-induced pulmonary inflammation is a well-known and well documented animal model for ARDS (see, for example, Matute-Bello et al., 2011, Am. J. Respir. Cell Mol. Biol. 44, 725-738; Orfanos et al., 2004, Intensive Care Med. 30, 1702-1714; Tsushima et al., 2009, Intern. Med. 48, 621-630).

In an LPS-induced animal model of ARDS, candidate compounds can be assessed for efficacy by measuring the extent of inflammation in the lungs of the animal relative to a suitable control (i.e., placebo). Inflammation in the lungs can be assessed by measuring cell counts from bronchoalveolar lavage (BAL) and levels of total protein or pro-inflammatory cytokines in BALF and lung parenchymal homogenates. LPS-induced permeability in the lung (i.e. extent of acute lung injury) can also be measured.

Influenza A virus (IAV) lung infection in C57BL/6 mice can largely reproduce the immunopathological features of SARS-COV-2 infection in hACE2 transgenic mice (see e.g., Winkler E S et al., 2020, Nat Immunol 21(11): 1327-1335).

In one example, therapeutic efficacy can be assessed in an IAV induced viral pneumonia animal model. For example, an IAV model that causes hyper-inflammation and viral pneumonia. In an IAV viral pneumonia model, candidate compounds can be assessed for efficacy by measuring the extent of inflammation in the lungs of the animal relative to a suitable control (i.e., placebo). Inflammation in the lungs can be assessed by measuring cell counts from BAL and levels of total protein or pro-inflammatory cytokines in BALF and lung parenchymal homogenates.

In some examples, assessing the therapeutic efficacy of a compound comprises detecting and/or quantifying the level of expression of a biomarker in the subject. Suitable biomarkers for assessing efficacy of treating ARDS include G-CSF, plasminogen activator inhibitor-1 (PAI-1), D-dimer, neutrophil elastase, soluble receptor for AGE (sRAGE), interferon gamma (IFN-γ), interleukin 1β (IL-1β), IL-2, IL-4, IL-6, IL-8, IL-10, IL-12p70, IL-13, and tumor necrosis factor alpha (TNF-α).

Detecting and/or quantifying biomarkers can be performed by any method known in the art. For instance, in one example, the levels of biomarkers are assessed using mass spectrometry. The mass spectrometry may be performed in conjunction with ultra-performance liquid chromatography (UPLC), high-performance liquid chromatography (HPLC), gas chromatography (GC), gas chromatography/mass spectroscopy (GC/MS), and UPLC, for example. Other methods of assessing levels of biomarkers include biological methods, such as but not limited to ELISA assays, Western Blot and multiplexed immunoassays etc. Other techniques may include using quantitative arrays, PCR, Northern Blot analysis. To determine levels of components or factors, it is not necessary that an entire component, e.g., a full length protein or an entire RNA transcript, be present or fully sequenced. In other words, determining levels of, for example, a fragment of protein being analyzed may be sufficient to conclude or assess that the level of the biomarker being analyzed is increased or decreased. Similarly, if, for example, arrays or blots are used to determine component levels, the presence/absence/strength of a detectable signal may be sufficient to assess levels of biomarkers.

To assess levels of biomarkers, a sample may be taken from the subject. The sample may or may not processed prior assaying levels of the components of the biomarker profile. For example, whole blood may be taken from an individual and the blood sample may be processed, e.g., centrifuged, to isolate plasma or serum from the blood. The sample may or may not be stored, e.g., frozen, prior to processing or analysis.

Biological samples that may be tested in a method of the invention include whole blood, blood serum, plasma, tracheal aspirate, BALF, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

### Compositions

In some examples, a CD131-binding compound as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, intrapolyp and intracranial injection or infusion techniques.

Methods for preparing a CD131-binding compound into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of a CD131-binding compound dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a CD131-binding compound of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a CD131-binding compound of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may he used to deliver a CD131-binding compound of the present disclosure.

WO2002/080967 describes compositions and methods for administering aerosolized compositions comprising antibodies for the treatment of respiratory conditions, which are also suitable for administration of compounds in accordance with the methods of the present disclosure.

### Combination Therapies

In one example, a compound of the present disclosure is administered in combination with another therapy useful for treating or preventing ARDS, either as combined or additional treatment steps or as additional components of a therapeutic formulation.

In some examples, the other therapy is one that is commonly used to treat or prevent ARDS. Contemplated therapies for ARDS, in combination with the methods of the disclosure, include treatments that involve decreasing lung inflammation, decreasing septal edema, decreasing alveolar and/or endothelial inflammation, treating the underlying cause of ARDS, and/or alleviating another symptom of the ARDS. The other therapy may comprise administration of a compound, cell or other molecule, and/or the other therapy may comprise physical or mechanical forms of therapy, for example artificial ventilation and prone positioning.

In some examples, the other therapy comprises prone positioning, fluid management, oxygenation, artificial ventilation (including newer modes of mechanical ventilation including, but not limited to, high frequency oscillatory ventilation), a glucocorticoid, a surfactant, inhaled nitric oxide, an antioxidant, a protease inhibitor, a recombinant human activated protein C, a β2-agonist, lisofylline, a statin, inhaled heparin, a diuretic, a sedative, an analgesic, a muscle relaxant, am anti-viral, an antibiotic, inhaled prostacyclin, inhaled synthetic prostacyclin analog, ketoconazole, alprostadil, keratinocyte growth factor, beta-agonists, human mAb against TS factor 7a, interferon receptor agonists, insulin, perfluorocarbon, budesonide, recombinant human ACE, recombinant human CC10 protein, tissue plasminogen activator, human mesenchymal stem cells, or nutritional therapy. In other examples of combination therapy, the other therapy is a glucocorticoid, such as, for example, methylprednisolone, dexamethasone, prednisone, prednisolone, betamethasone, triamcinolone, triamcinolone acetonide hudesonide, and beclometasone; beta-agonists, such as, for example, albuterol; lisofylline; rosuvastatin, inhaled heparin; inhaled nitric oxide; recombinant human activated protein C; NSAIDS, such as, for example, ibuprofen; naproxen, and acetaminophen; cisatracurium besylate; procysteine; acetylcysteine; inhaled prostacyclin; ketoconazole; alprostadil; keratinocyte growth factor; human mAb against TS factor 7a; insulin; perfluorocarbons, recombinant human ACE; recombinant human CC10 protein; tissue plasminogen activator; human mesenchymal stem cells; or nutritional therapy such as a combination of omega-3 fatty acids, antioxidants, and γ-linolenic acids with isocaloric foods and extracorporeal membrane oxygenation (ECMO).

NSAIDS include, but are not limited to, aspirin, acetaminophen, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, nabumetone, diclofenac, piroxicam, meloxicam, tenoxicam, droxicam, lornoxicam, isoxicam, mefenamic acid, meclofenamic acid, flufenamic acid, tolfenamic acid, celecoxib, parecoxib, etoricoxib, lumiracoxib, and firocoxib.

Analgesics include, but are not limited to, NSAIDS and opioids (narcotics). Opioids include, but are not limited to, dextropropoxyphene, codeine, tramadol, tapentadol, anileridine, alphaprodine, pethidine, hydocodone, morphine, oxycodone, methadone, diamorphine, hydromorphone, oxymorphone, levorphanol, 7-hydroxymitragynine, buprenorphine, fentanyl, sufentanil, bromadol, etorphine, dihydroetoiphine, and carfentanil.

Glucocorticoids include, but are not limited to, hydrocortisone, cortisone, prednisone, prednisolone, methylprednisolone, dexamethasone, betamethasone, triamcinolone, beclometasone, or fludrocortisones.

In some examples, the other therapy is a standard of care therapy. Standard of care therapies that are commonly used to treat or prevent ARDS include treatment of the underlying condition (e.g., infection), mechanical or noninvasive ventilation, fluid and hemodynamic therapy, prone positioning, treatment of opportunistic infection, nutrition, and pharmacologic therapy. For example, the Faculty of Intensive Care Medicine (FICM) and intensive Care Society (ICS) recently released guidelines for standard of care therapy for adult patients with ARDS (Griffiths et al., 2019, BMJ Open Resp Res 6:e000420). Where mechanical ventilation is required, the use of low tidal volumes (<6 ml/kg ideal body weight) and airway pressures (plateau pressure <30 cmH2O) was recommended. For patients with moderate/severe ARDS (PaO₂/FiO₂ ratio of less than or equal to 150 mmHg), prone positioning was recommended for at least 12 hours per day. The use of a conservative fluid management strategy was suggested for all patients, whereas mechanical ventilation with high positive end-expiratory pressure and the use of the neuromuscular blocking agent cisatracurium. for 48 hours was suggested for patients with ARDS with arterial oxygen partial pressure to fractional inspired oxygen (PaO₂/FiO₂) ratios less than or equal to 200 mmHg and 150 mmHg, respectively. Extracorporeal membrane oxygenation was suggested as an adjunct to protective mechanical ventilation for patients with very severe ARDS. The methods of the disclosure can be performed in combination with any of the above therapies for treatment or prevention of ARDS.

In sonic examples, the other therapy is one that is used to treat the underlying cause of ARDS. For instance, in one example, the other therapy comprises administration of an antiviral or antibiotic (e.g., where the underlying cause of ARDS is an infection). In one example, the other therapy comprises administration of remdesivir.

In one example, the compound that binds to CD131 is administered simultaneously with the other therapy. In one example, the compound that hinds to CD131 is administered before the other therapy. In one example, the compound that binds to CD131 is administered after the other therapy.

In some examples, the compound that binds to CD131 is administered in combination with a cell. In some examples, the cell is a stein cell, such as a mesenchymal stein cell. In some examples, the compound that binds to CD131 is administered in combination with a gene therapy.

### Dosages and Timing of Administration

Suitable dosages of a CD131-binding compound of the present disclosure will vary depending on the specific CD131-binding compound, the condition to he treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically or prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In sonic examples, a method of the present disclosure comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will he able to determine appropriate dosages depending on these and other factors. Accordingly, this term is not to be construed to limit the present disclosure to a specific quantity, e.g., weight or amount of protein(s), rather the present disclosure encompasses any amount of the CD131-binding compound(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific CD131-binding protein(s) administered and/or the particular subject and/or the type or severity or level of condition an/or predisposition (genetic or otherwise) to the condition. Accordingly, this term is not to be construed to limit the present disclosure to a specific quantity, e.g., weight or amount of CD131-binding compound.(s), rather the present disclosure encompasses any amount of the CD131-binding protein(s) sufficient to achieve the stated result in a subject.

For in vivo administration of the CD131-binding compound described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the CD131-binding compound is administered at an initial (or loading) dose of between about 1 mg/kg to about 30 mg/kg, such as from about 1 mg/kg to about 10 mg/kg, or about 1 mg/kg or about 2 mg/kg or 5 mg/kg. The CD131-binding compound can then be administered at a lower maintenance dose of between about 0.01 mg/kg to about 2 mg/kg, such as from about 0.05 mg/kg to about 1 mg/kg, for example, from about 0.1 mg/kg to about 1 mg/kg, such as about 0.1 mg/kg or 0.5 mg/kg or 1 mg/kg. The maintenance doses may be administered every 7-30 days, such as, every 10-15 days, for example, every 10 or 11 or 12 or 13 or 14 or 15 days.

In some examples, the CD131-binding compound is administered at a dose of between about 0.01 mg/kg to about 50 mg/kg, such as between about 0.05 mg/kg to about 30 mg/kg, for example, between about 0.1 mg/kg to about 20 mg/kg, for example, between about 0.1 mg/kg to about 10 mg/kg, such as between about 0.1 mg/kg to about 2 mg/kg. For example, the CD131-binding compound is administered at a dose of between about 0.01 mg/kg to about 5 mg/kg, such as from about 0.1 mg/kg to about 2 mg/kg, such as about 0.2 mg/kg or 0.3 mg/kg or 0.5 mg/kg or 1 mg/kg or 1.5 mg/kg (e.g., without a higher loading dose or a lower maintenance dose). In some examples, numerous doses are administered, e.g., every 7-30 days, such as, every 10-22 days, for example, every 10-15 days, for example, every 10 or 11 or 12 or 13 or 14 or 15 or 16 or 17 or 18 or 19 or :20 or 21 or 22 days. For example, the CD131-binding compound is administered every 7 days or every 14 days or every 21 days.

In some examples, at the time of commencing therapy, the mammal is administered the CD131-binding compound on no more than 7 consecutive days or 6 consecutive days or 5 consecutive days or 4 consecutive days.

In the case of a mammal that is not adequately responding to treatment, multiple doses in a week may be administered. Alternatively, or in addition, increasing doses may be administered.

In another example, for mammals experiencing an adverse reaction, the initial (or loading) dose may be split over numerous days in one week or over numerous consecutive days.

Administration of a CD131-binding compound according to the methods of the present disclosure can be continuous or intermittent, depending, for example, on the recipient's physiological condition, whether the purpose of the administration is therapeutic or prophylactic, and other factors known to skilled practitioners. The administration of a CD131-binding compound may he essentially continuous over a preselected period of time or may he in a series of spaced doses, e.g., either during or after development of a condition.

### Kits

Another example of the disclosure provides kits containing compounds useful for the treatment or prevention of ARDS as described above.

In one example, the kit comprises (a) a container comprising a compound that hinds to CD131 as described herein, optionally in a pharmaceutically acceptable carrier or diluent; and (b) a package insert with instructions for treating, preventing, or reducing an effect of ARDS in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating or preventing the ARDS and may have a sterile access port (for example, the container may be an intravenous solution bag or a vial having a stopper pierceable by a hypodermic injection needle). At least one active agent in the composition is the compound that binds to CD131. The label or package insert indicates that the composition is administered to a subject eligible for treatment, e.g., one having or at risk of developing ARDS, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Anti-CD131/βc and βIL3 Chimeric Monoclonal Antibody Reduces Neutrophil and Macrophage Accumulation in the Lung

### Antibodies

“Chimeric mAb” is a chimeric mouse monoclonal antibody comprising a rat Fab region which recognizes the extracellular region of the mouse CD131/βc and βIL3 cytokine receptor subunits and inhibits signaling through these receptors and a mouse IgG1 Fc. An isotype control antibody was used for comparison (muBM4-muGIK RP3204 or “BM4”).

### Acute Respiratory Distress Syndrome (ARDS) Model

Wild-type (c57BL/6 mice aged 8-12 weeks) mice were used. Mouse colonies were obtained from the Animal Resources Centre (ARC) (Canning Vale, Western Australia, Australia). Eight-week-old to twelve-week old female mice used for the experiments performed in compliance with the ethical guidelines of the National Health and Medical Research Council of Australia.

ARDS model: Mice were anesthetised and intubated by intratracheal (i.t.) installation of 3 μg Escherichia coli LPS (LPS; 0111:B4, Sigma-Aldrich) into the lungs using a cannula attached to a 50 ml Hamilton syringe into the mouth and trachea. Mice were terminated 24 h after LPS intubation by lethal injection of pentobarbital.

Evaluation in ARDS model: To assess the effects of prophylactic and therapeutic administration of chimeric mAb in the ARDS model, mice were administered 50 mg/kg chimeric mAb or BM4 intravenously (i.v.) either 24 h prior to (prophylactic) or 6 h after (therapeutic) the intubation of LPS. Broncho-alveolar lavage fluid (BALF) was obtained by cannulating the trachea with a 20G catheter. Both lungs were lavaged three times (each aliquot 0.4 ml PBS) with average return of 0.9-1.1 ml/mouse of BALF.

### Cellular Analysis of BALF

Total cell number in BALF was determined by counting with a haemocytometer. Differential counts were done by cytocentrifuged preparations (cytosine 4; Thermo Scientific), fixed and stained with Geimsa (Sigma). Differential counts were based on counts of 200 cells using standard morphologic criteria to classify the cells as neutrophils, macrophages or lymphocytes. Counts were performed by a single observer blinded to the treatment groups.

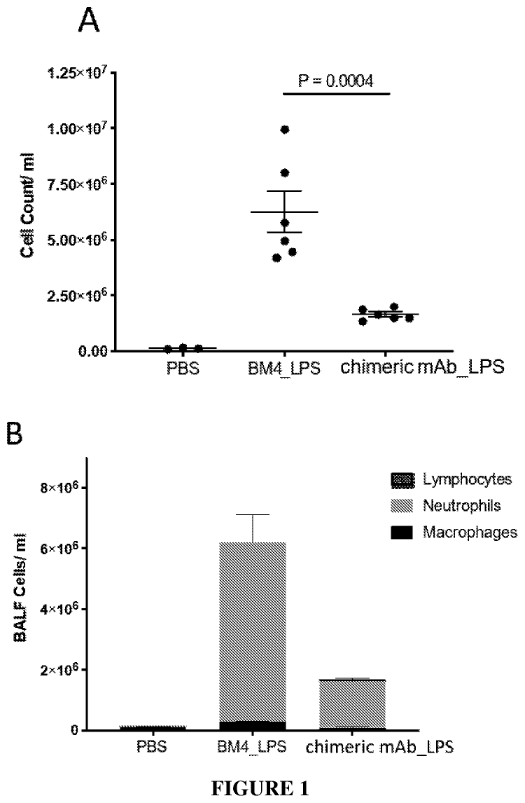

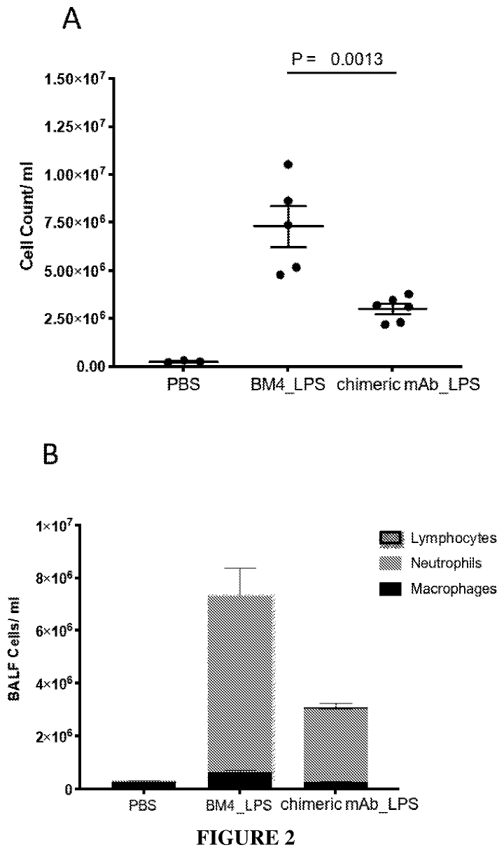

Significant reduction in the cell numbers in the BALF was observed in the ARDS models prophylactically or therapeutically administered chimeric mAb compared to BM4 groups (FIGS. 1A and 2A). This reduction correlates with the reduction of macrophages and neutrophils observed in the BALF following prophylactic or therapeutic administration of chimeric mAb (FIGS. 1B and 2B).

### Example 2: Anti-CD131/βc and βIL3 Chimeric Monoclonal Antibody Inhibits GM-CSF and IL-3 Induced Proliferation of FDCP1 Cells

FDCP1 mouse bone marrow-derived cell line was starved of mIL-3 for 3 h in 10% fetal calf serum (FCS) and plated at a density of 1×10⁴ cells/well in 200 μl RPMI/10% FCS in a dose response of either isotype control monoclonal antibody (isotype control mAb) or chimeric monoclonal antibody (chimeric mAb) as used in Example 1. After 1 h pre-incubation with isotype control mAb or chimeric mAb, an ED₅₀ dose of mIL3 (0.54 ng/ml) or mGM-CSF (0.022 ng/ml) was added to each well.

Proliferation was measured 48 h after incubation with mIL3 or mGM-CSF. The FDCP1 cells were incubated for 1 h at 37° C./5% CO₂ in 20 μl/well of Cell Titre96 Aqueous One Solution (Promega). Optical density (OD) reading at 490 nm to 690 nm was performed and data imputed into Prism.

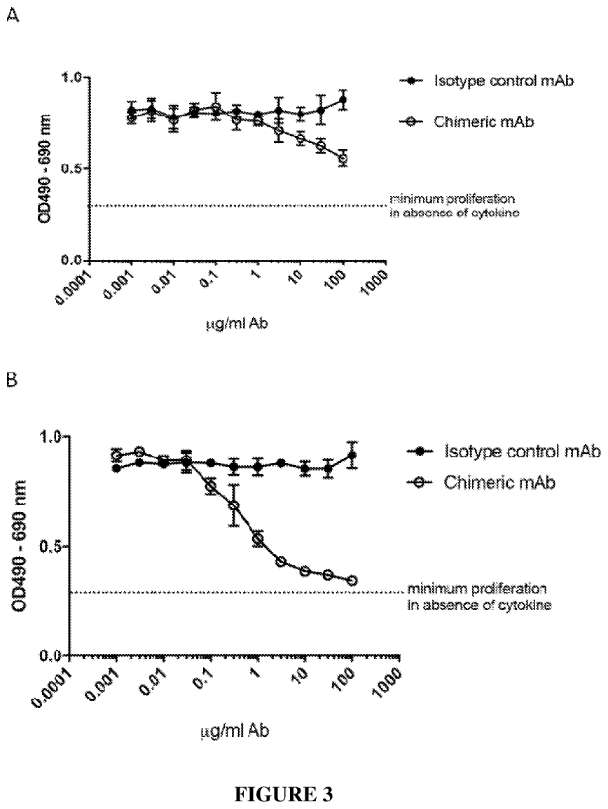

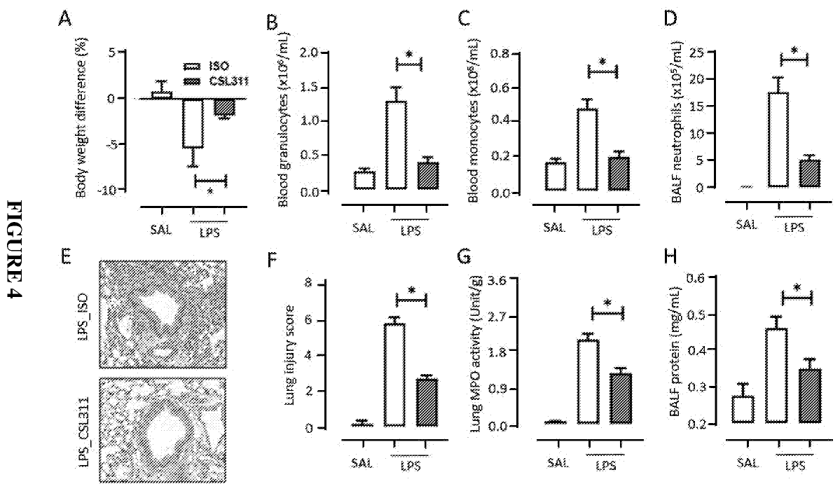

FIG. 3 demonstrates that chimeric mAb reduces proliferation of FDCP1 cells by inhibiting GM-CSF and IL-3 signaling in a dose dependent manner.

### Example 3: CSL311 Reduces Cellular Inflammation, Reduces Tissue Pathology and Increases Blood Oxygenation in a hβcTg Mouse Model of Acute Respiratory Distress Syndrome (ARDS)

### Antibodies

CSL311 (ahu9A2-G4pK-VR24-29) is a human antibody targeting the common cytokine binding site (site 2) of the human βc (CD131) homodimer. Heavy chain amino acid sequence is provided in SEQ ID NO: 14 and light chain amino acid sequence is provided in SEQ ID NO: 15. A human IgG4 (chBM4-G4pK) isotype control antibody was used for comparison.

### Acute Respiratory Distress Syndrome (ARDS) Model

To establish the therapeutic potential of CSL311 for treatment of ARDS, lung leukocyte numbers and markers of lung injury were examined in a transgenic mouse model expressing human βc receptor. The transgenic mice, referred to as “hβcTg”, were homozygous for (i) a human βc receptor transgene and (ii) knock out for both endogenous beta subunits (βc^{-/-}/β_{IL-3}_{^{-/-}}). ARDS was induced in hβcTg mice by intranasal instillation of 10 μg Escherichia coli LPS (LPS; 026:B6, Sigma-Aldrich) for 24 h. Mice were treated with CSL311 (50 mg/kg) or isotype control mAb intravenously 3 h prior to LPS challenge.

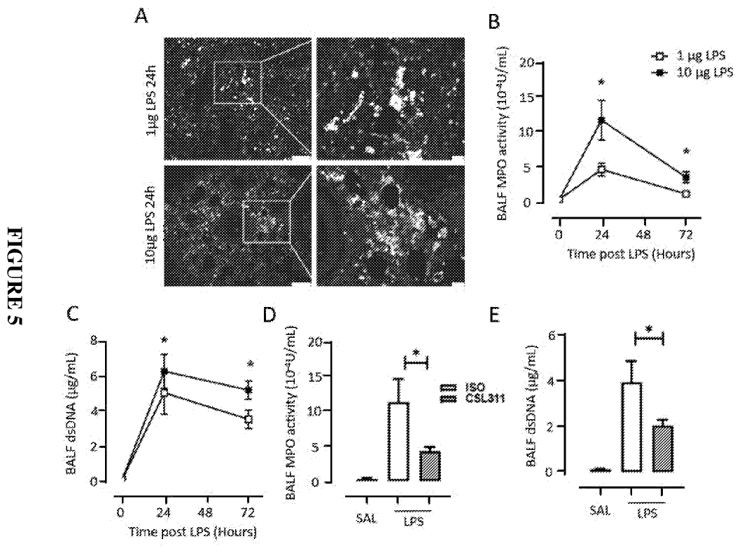

Direct lung instillation of LPS resulted in significant body weight reduction in hβcTg mice challenged with LPS which was significantly reduced with the administration of CSL311 (FIG. 4A). LPS challenge markedly increased blood neutrophil and monocyte numbers in hβcTg mice, which were reduced to control levels with CSL311 administration (FIGS. 4B and 4C). Neutrophils migrated into the broncho-alveolar lavage (BAL) airway compartment in response to LPS challenge which was significantly reduced by approximately 70% with CSL311 administration (FIG. 4D). Histological scoring of lung inflammation revealed that CSL311 significantly reduced lung injury caused by acute LPS challenge in hβcTg mice (FIGS. 4E and 4F). Peak lung myeoloperoxidase (MPO) levels also significantly decreased consistent with a marked reduction in lung tissue leukocyte infiltration (FIG. 4G). In addition, CSL311 significantly reduced peak oedoma caused by LPS challenge as assessed by quantifying total protein levels in BALF (FIG. 4H).

The release of neutrophil extracellular traps (NETs) which have been associated with NETopathic tissue damage in response to acute injury or infection was observed with acute LPS challenge of hβcTg mice (FIG. 5A). FIG. 5A shows staining of lung tissue sections of LPS challenged hβcTg mice with antibodies against MPO and citrullinated histone H3. NETs formation was significantly increased at the higher dose of LPS challenge (10 μg) in hβcTg mice (FIG. 5A). NETs were also quantified in the BALF of LPS challenged hβcTg mice demonstrating peak MPO and dsDNA level 24 h after LPS challenge (10 μg) (FIGS. 5B and 5C). The peak MPO and dsDNA levels were significantly reduced with administration of CSL311 prior to LPS challenge of hβcTg mice (FIGS. 5D and 5E).

To determine whether CSL311 can assist in maintaining lung function in response to ARDS, blood oxygenation was examined in hβcTg mice. ARDS was induced in hβcTg mice by intranasal instillation of 20 μL of 5 μg/mL LPS. Mice were treated with CSL311 (50 mg/kg) or isotype control mAb (50 mg/kg) intravenously 30 minutes prior to LPS challenge. Mice treated with intranasal PBS were used to determine blood oxygenation in the absence of LPS challenge. The percentage of blood oxygenation was monitored at 16 h, 48 h and 72 h post LPS or PBS administration using a MouseSTAT® Jr. Pulse Oximeter & Heart Rate Monitor (Kent Scientific).

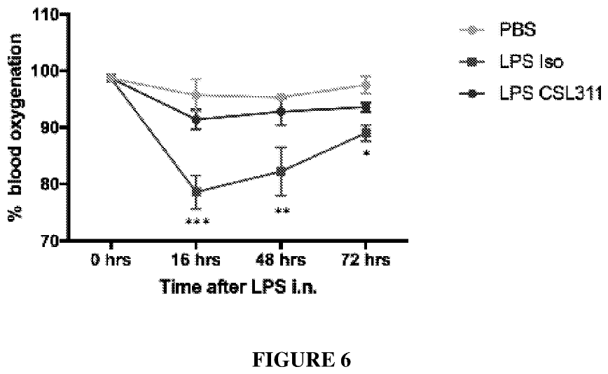

LPS administration to hβcTg mice resulted in acute reduction in blood oxygenation that peaked at approximately 16 h and began to resolve thereafter (FIG. 6, LPS-ISO group). Administration of CSL311 significantly protected hβcTg mice against LPS-induced reduction in blood oxygenation at 16 h, 48 h and 72 h compared to isotype control antibody (FIG. 6). Together, these data demonstrate the effectiveness of targeting the βc receptor (CD131) for the treatment and prevention of ARDS.

### Example 4: CSL311 Reduces Immunopathology and Hyperinflammation in a Pre-Clinical Mouse Model of Viral Pneumonia

### Viral Pneumonia Model

Viral pneumonia was induced in transgenic hβcTg mice. Briefly, hβcTg mice were intranasally infected with influenza A virus (IAV, HKx31, H3N2 strain, 10⁴ PFU) under light isoflurane anaesthesia.

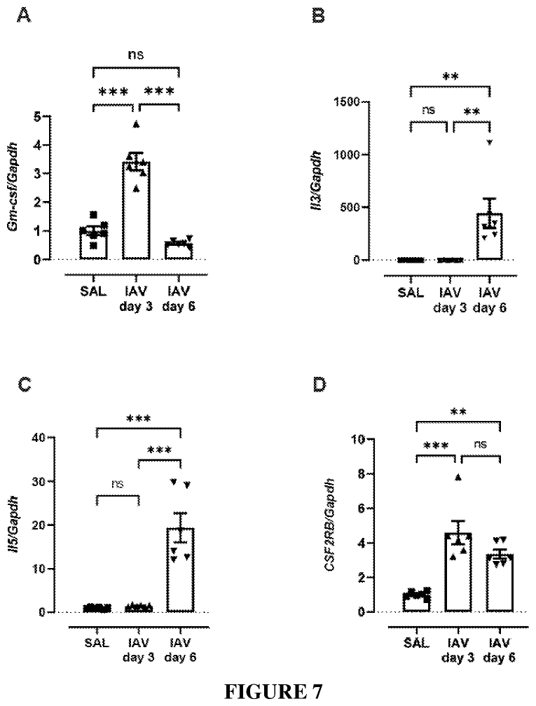

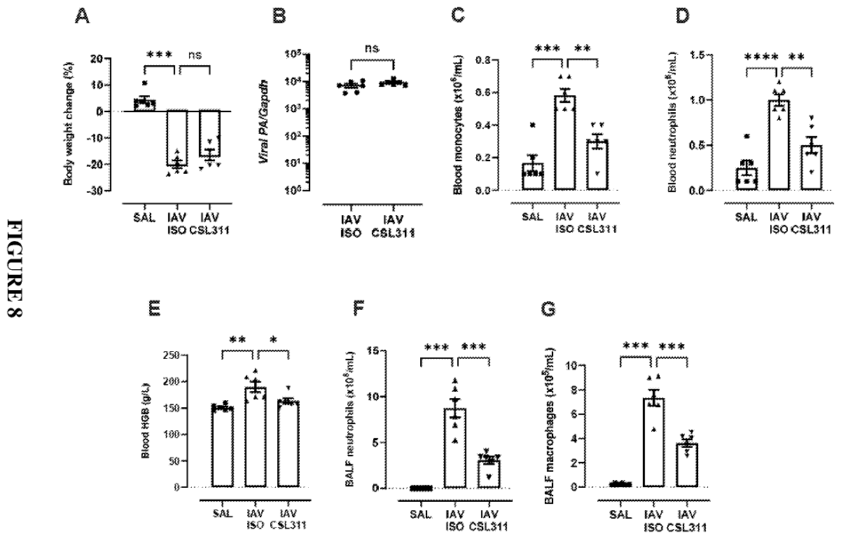

In hβcTg mice, Gm-csf expression in lung tissue significantly increased during the early phase of IAV infection (day 3) and declined at day 6 (FIG. 7A). In contrast, Il3 and Il5 expression peaked at the later day 6 time point (FIG. 7B and 7C).

Expression of the βc receptor transcript (CSF2RB) was significantly increased at both day 3 and day 6 post IAV infection, consistent with persistent tissue infiltration of βc receptor-expressing inflammatory cells during acute viral infection (FIG. 7D).

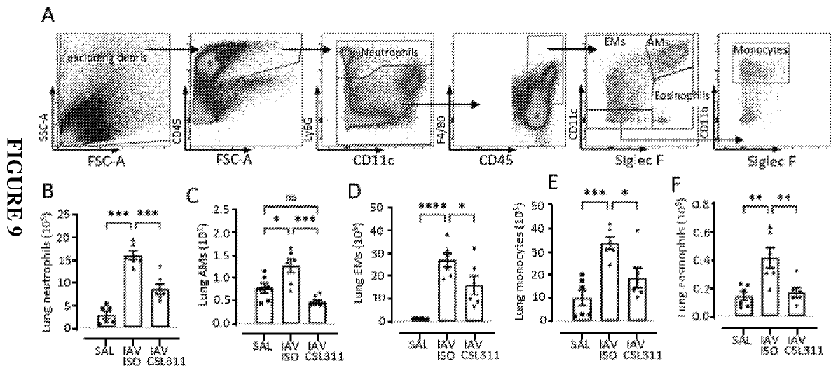

Four days after IAV infection, mice were treated with a single dose of CSL311. (50 mg/kg) or isotype control mAb (50 mg/kg) via intravenous injection. On day 6, mice infected with IAV displayed a significant reduction in body weight and this was not altered by CSL311 treatment (FIG. 8A). Lung viral loads were determined by RTqPCR on viral polymerase A subunit (PA) gene and detected high levels of viral RNA in IAV-infected mice. Levels were identical in CSL311 treated mice (FIG. 8B). Blood granulocytosis and monocytosis induced by IAV infection was significantly reduced by βc receptor blockage with CSL311 treatment (FIG. 8C & 8D). In addition, increased levels of blood haemoglobin (HGB), which is likely to be secondary to IAV-induced hypoxemia, was completely prevented by CSL311 treatment (FIG. 8E). Neutrophil (FIG. 8F) and macrophage (FIG. 8G) numbers in the bronchoalveolar lavage (BAL) compartment were markedly increased in IAV-infected mice and CSL311 significantly reduced both of these myeloid cell populations. In addition, IAV infection caused significant haemorrhaging throughout the lung lobes as characterised by the appearance of darkened lung lobes that are plum in colour, as opposed to lungs from saline treated mice that were pink in colour. CSL311 treatment was associated with a reduction in haemorrhagic regions throughout the lung lobes.

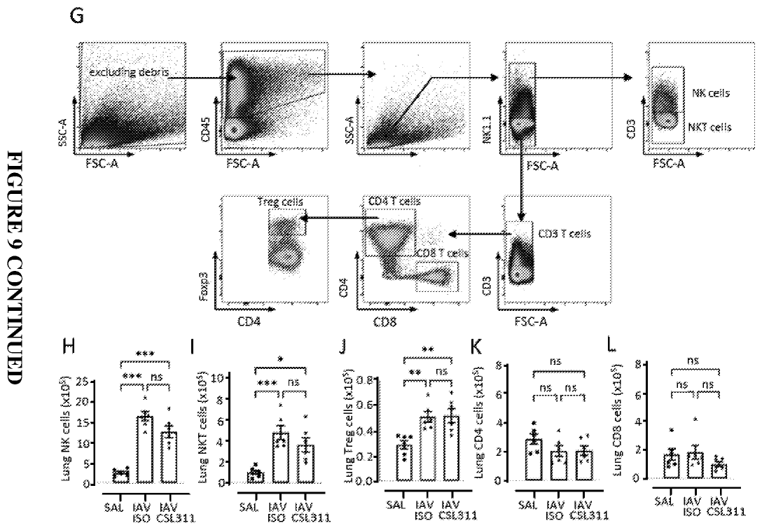

To further characterise immunological changes in the lungs following CSL311 treatment of IAV infected hβcTg mice, myeloid (FIG. 9A) and lymphoid cells (FIG. 9G) were analysed by flow cytometry. Briefly, following BAL, the superior lobes of the lungs were finely minced and digested in Liberase™ (Sigma, US) at 37° C. with constant shaking. Single cell suspension was prepared by passing the digested tissue through a 25G needle and then a 40 μm cell strainer. Lung cells were pelleted, and red blood cells were lysed with ACK lysis buffer. After blocking with CD16/CD32 antibody, cells were then stained with a myeloid cell antibody cocktail containing FITC-CD45, PE-Siglec F, APC-F4/80, eFluor 450-CD11b, PE/Cy7-CD11c, PerCp/eFluor710-Ly6G and LIVE/DEAD™ Fixable Yellow Dead Cell Stain, or a lymphoid cell antibody cocktail containing APC/eFluor 780-CD45, PerCP/Cyanine5.5-CD3e, PE/Cy7-NK-1.1 (BD, US), PE-CD8a (BioLegend, US), PE/eFluor 610-CD4, Alexa Fluor 488-FOXP3 (BioLegend, US) and LIVE/DEAD™ Fixable Violet Dead Cell Stain as previously described 23, 26, 27. A Foxp3/Transcription Factor Staining Buffer Set was used for cell permeabilization before Foxp3 staining. After staining, cells were fixed with an IC Fixation Buffer before analysed on a BD FACSAria.

Lung neutrophils (FIG. 9B) and macrophages (FIG. 9C-E) were significantly reduced in in IAV-infected mice following CSL311 treatment, consistent with changes observed in the blood and BAL. The reduction in macrophage numbers were attributed to the normalisation of alveolar macrophages (AMs, FIG. 9C) and suppression of monocyte derived exudative macrophages (EMs, FIG. 9D) and blood monocytes (FIG. 9E) recruited into the lungs. Lung eosinophils numbers, previously reported to increase in IAV-infected mice, were also increased in IAV-infected hβcTg mice and βc receptor antagonism with CSL311 markedly reduced this response (FIG. 9F). In contrast to myeloid cells, lymphocyte subsets that confer vital protection against viral infections do not directly respond to βc cytokines. A marked expansion of natural killer (NK) cells (FIG. 9H) and NKT cells (FIG. 9I) was observed at day 6 post IAV infection, which was not significantly altered by CSL311 treatment. Immunosuppressive regulatory T (Treg) cells were increased by IAV infection in a manner that was not affected by CSL311 treatment (FIG. 9J). Lung CD4 and CD8+ T cells were not increased at day 6 post IAV infection and CSL311 treatment did not further alter their respective numbers (FIG. 9K & 9L).

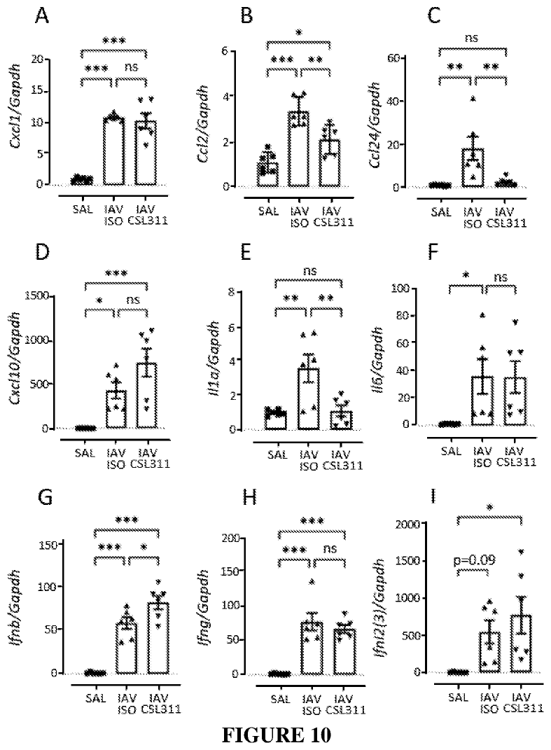

At a molecular level, lung expression of the neutrophil chemokine Cxcl1 gene, the monocyte/macrophage chemokine Ccl2 gene and the eosinophil chemokine Cc124 gene was significantly increased in IAV-infected mice (FIG. 10A-C). CSL311 treatment did not reduce Cxcl1 gene expression but did significantly reduce Ccl2 and Ccl24 gene expression. These reductions are consistent with lung macrophages being a major cellular source of CCL2 and CCL24 during IAV infection, which are reduced by CSL311 treatment. Levels of Cxcl10 and Cxcl1 gene expression were increased in IAV-infected mice in a manner that was not altered by CSL311 treatment (FIG. 10D). Cxcl1 and Cxcl10 can be produced by various cell types including fibroblasts and endothelial cells, which are not targets for βc antagonism. Gene expression of the inflammatory cytokine Il1α was increased in the lungs of IAV-infected mice and βc receptor blockade completely normalised Il1β gene levels (FIG. 10E). IL-1R signalling can regulate expression of neutrophil adhesion molecules and the reduction in IL-1α may contribute to reduced neutrophilic inflammation in the lungs. Il6 gene expression was also increased in IAV-infected mice and its levels were not reduced in CSL311 treated mice (FIG. 10F). Interferons constitute the first line of anti-viral defence and all three types of interferons were upregulated in IAV-infected lungs (FIG. 10G-I). Type II interferons (Ifng) gene expression levels were not altered by CSL311 treatment, consistent with cellular sources (NK/NKT cells and cytotoxic T cells) not being altered by CSL311 treatment. IAV-induced gene expression of type I (Ifnb) and type III interferons (Ifnl2/3) was also preserved in CSL311-treated mice (FIG. 10G & I), which suggests that their cellular sources (plasmacytoid dendritic cells (pDCs) and lung epithelia) are functionally competent in the background of βc receptor antagonism.

1. A method for preventing or treating acute respiratory syndrome (ARDS) in a subject, the method comprising administering a compound that binds to CD131 and neutralizes signaling by interleukin (IL) 3, IL-5, and granulocyte-macrophage colony stimulating factor (GM-CSF) to the subject.

2. The method of claim 1, wherein the ARDS is associated with one or more of the following:
a) an infection;
b) inhalation or aspiration of a foreign substance;
c) a physical trauma;
d) an inflammatory disease; and/or
e) pneumonia.

3. The method of claim 1, wherein the ARDS is associated with a viral infection and/or a coronavirus infection and/or a severe acute respiratory syndrome coronavirus (SARS-COV) infection and/or a SARS-CoV-2 infection and/or a coronavirus disease 2019 (COVID-19).

4-7. (canceled)

8. The method of claim 1, wherein the subject has interstitial pneumonia.

9. The method of claim 1, wherein administration of the compound that binds to CD131 and neutralizes signaling by IL-3, IL-5 1, and GM-CSF reduces, or prevents an increase in, one or more or all of the following:
a) amount of neutrophils in the subject's blood,
b) amount of monocytes in the subject's blood,
c) neutrophil accumulation in the subject's lung,
d) macrophage accumulation in the subject's lung,
e) inflammation of the subject's lung,
f) oedema of the subject's lung,
g) NETosis in the subject's lung,
h) myeloperoxidase activity in the bronchoalveolar fluid of the subject's lung,
i) amount of protein in the bronchoalveolar fluid of the subject's lung,
j) amount of dsDNA in the bronchoalveolar fluid of the subject's lung, and/or
k) a lung injury score in the subject.

10. The method of claim 1, wherein administration of the compound that binds to CD131 and neutralizes signaling by IL-3, IL-5, and GM-CSF prevents a decrease in percentage blood oxygenation in the subject.

11. The method of claim 1, wherein the compound that binds to CD131 and neutralizes signaling by IL-3, IL-5, and GM-CSF inhibits
a) GM-CSF-induced proliferation of TF-1 cells with an IC50 of at least 100 nM; and/or
b) IL-5-induced proliferation of TF-1 cells with an IC50 of at least 100 nM; and/or
c) IL-3-induced proliferation of TF-1 cells with an IC50 of at least 100 nM.

12. The method of claim 1, wherein the compound that binds to CD131 and neutralizes signaling by IL-3, IL-5, and GM-CSF is a protein comprising an antigen binding site that binds to CD131.

13. The method of claim 12, wherein the K_{D} of the protein for a polypeptide comprising a sequence set forth in SEQ ID NO: 5 is about 10 nM or less, when the polypeptide is immobilized on a solid surface and the K_{D} is determined by surface plasmon resonance.

14. The method of claim 1, wherein the compound that binds to CD131 and neutralizes signaling by IL-3, IL-5, and GM-CSF is a protein comprising a Fv.

15. The method of claim 14, wherein the protein comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

16. The method of claim 12, wherein the protein is a single domain antibody (sdAb).

17. The method of claim 12, wherein the protein comprises an antibody variable region that binds to CD131 and competitively inhibits the binding of antibody 9A2-VR24.29 comprising a heavy chain variable region (V_{H}) comprising a sequence set forth in SEQ ID NO: 6 and a light chain variable region (V_{L}) comprising a sequence set forth in SEQ ID NO: 18 to CD131.

18. The method of claim 12, wherein the antigen binding site:
(a) binds to an epitope within Site 2 of CD131; and/or
(b) binds to an epitope formed upon dimerization of two CD131 polypeptides; and/or
(c) binds to residues within domain 1 of a CD131 polypeptide and residues within domain 4 of another CD131 polypeptide.

19. (canceled)

20. (canceled)

21. The method of claim 18, wherein the residues within domain 1 of CD131 comprise residues in the region of 101-107 of SEQ ID NO: 1 and/or the residues within domain 4 of CD131 comprise residues in the region of 364-367 of SEQ ID NO: 1.

22. The method of claim 12, wherein the protein comprises:
(a) an antibody variable region comprising a VH comprising three CDRs of a VH comprising an amino acid sequence set forth in SEQ ID NO: 6 and a VL comprising three CDRs of a VL comprising an amino acid sequence set forth in SEQ ID NO: 18; and/or
(b) a VH comprising an amino acid sequence set forth in SEQ ID NO: 6 and a VL comprising an amino acid sequence set forth in SEQ ID NO: 18; and/or
(c) a heavy chain comprising an amino acid sequence set forth in SEQ ID NO: 8 and a light chain comprising an amino acid sequence set forth in SEQ ID NO: 9.

23. (canceled)

24. (canceled)

25. The method of claim 1, wherein the compound that binds to CD131 is administered in combination with a standard of care therapy.

26. The method of claim 25, wherein the standard of care therapy comprises one or more or all of the following:
a) prone positioning;
b) fluid management;
c) administration of nitric oxide;
d) administration of a neuromuscular blocking agent;
e) artificial ventilation;
f) extracorporeal membrane oxygenation;
g) administration of an antiviral agent or antibiotic; and/or
h) administration of remdesivir.

27. (canceled)

28. The method of claim 1, wherein the subject is at risk of developing ARDS and wherein the subject at risk of developing ARDS has pneumonia.


SEQUENCE LISTING (18 sequences)

SEQ ID NO: 1 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 2 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 3 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 4 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 5 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 6 - protein, 117 aa, Artificial Sequence
  Synthetic antibody sequence
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSS

SEQ ID NO: 7 - protein, 112 aa, Artificial Sequence
  Synthetic antibody sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTVAA

SEQ ID NO: 8 - protein, 10 aa, Artificial Sequence
  Synthetic antibody sequence
GFTFPWYRVH

SEQ ID NO: 9 - protein, 17 aa, Artificial Sequence
  Synthetic antibody sequence
SIRSSGGFPYYNYKVKG

SEQ ID NO: 10 - protein, 8 aa, Artificial Sequence
  Synthetic antibody sequence
FYDSFFDI

SEQ ID NO: 11 - protein, 11 aa, Artificial Sequence
  Synthetic antibody sequence
RASQGISSWLA

SEQ ID NO: 12 - protein, 7 aa, Artificial Sequence
  Synthetic antibody sequence
AASSLQS

SEQ ID NO: 13 - protein, 9 aa, Artificial Sequence
  Synthetic antibody sequence
QQANSFPIT

SEQ ID NO: 14 - protein, 444 aa, Artificial Sequence
  Synthetic antibody sequence
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, Artificial Sequence
  Synthetic antibody sequence
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 327 aa, Artificial Sequence
  Synthetic antibody sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 104 aa, Artificial Sequence
  Synthetic antibody sequence
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 108 aa, Artificial Sequence
  amino acid sequence of a Vl
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKR